(19) United States
(12) Patent Application Publication
(10) Pub. No.: US 2013/0005678 A1
(43) Pub. Date: Jan. 3, 2013

(54) Methods and Compositions for Treating or Ameliorating Cancer Using Gemcitabine-5'-Elaidate

(75) Inventors: Marit Liland Sandvold, Porsgrunn (NO); Finn Myhren, Porsgrunn (NO)

(73) Assignee: CLAVIS PHARMA ASA, Oslo (NO)

(21) Appl. No.: 13/583,982

(22) PCT Filed: Mar. 30, 2011
(86) PCT No.: PCT/IB11/01175

§ 371 Date: Sep. 11, 2012

Related U.S. Application Data
(60) Provisional application No. 61/319,149, filed on Mar. 30, 2010.
(60) Provisional application No. 61/388,763, filed on Oct. 1, 2010.

(51) Int. Cl. A61K 31/7068; A61P 35/02; A61P 35/00

(52) U.S. Cl. 514/49

(57) ABSTRACT

This invention provides methods and compositions for treating or otherwise ameliorating cancer in a subject, along with methods and compositions for measuring the levels of nucleoside transporters in a tumor and correlating this level to a predicated efficacy of a given anti-cancer drug regime, and methods and compositions for treating patients with low levels of hENT1 expression in cancer cells using a lipophilic gemcitabine analog such as gemcitabine-5′-elaidate.

### RELATED APPLICATIONS

This application claims the benefit of U.S. Provisional Application No. 61/319,149, filed Mar. 30, 2010, and U.S. Provisional Application No. 61/388,763, filed Oct. 1, 2010, the contents of each of which are hereby incorporated by reference in their entirety.

### FIELD OF THE INVENTION

This invention relates generally to methods and compositions for treating or otherwise ameliorating cancer in a subject, to methods and compositions for measuring the levels of nucleoside transporters in a tumor and correlating this level to a predicated efficacy of a given anti-cancer drug regime, and methods and compositions for treating patients with low levels of hENT1 expression in cancer cells using a lipophilic gemcitabine analog such as gemcitabine-5′-elaidate.

### BACKGROUND OF THE INVENTION

Nucleoside transporters are a group of membrane transport proteins which allow for movement of physiologic nucleosides through the plasma membrane. Nucleoside transporters are required for nucleotide synthesis in cells that lack de novo nucleoside synthesis pathways, and are also necessary for the uptake of cytotoxic nucleosides and nucleoside analog drugs used for cancer and viral chemotherapies. Many, but not all cancer cells, express nucleoside transporters.

There are two major classes of nucleoside transporters in human cells and tissues, the equilibrative nucleoside transporters (ENTs) and the concentrative nucleoside transporters (CNTs). The ENT family is also known as SLC29. There are four known ENTs, designated ENT1, ENT2, ENT3, and ENT4. ENTs are blocked by adenosine reuptake inhibitors such as dipyridamole and dilazep. The CNT family, also known as SLC28, has three members: CNT1, CNT2 and, also designated as CNT3SLC28A1, SLC28A2 and SLC28A3.

Human Equilibrative Nucleoside Transporter 1 (hENT1) is one example of a nucleoside transporter expressed in some cancer cells. hENT1 is encoded by the SLC29A1 gene. The gene encodes a transmembrane glycoprotein that localizes to the plasma and mitochondrial membranes and mediates the cellular uptake of nucleosides from the surrounding medium. The protein is categorized as an equilibrative (as opposed to concentrative) transporter that is sensitive to inhibition by nitrobenzylthioinosine (NBMPR).

Nucleoside transporters can move hydrophilic nucleoside anticancer drugs across the plasma membrane. Some examples of these drugs are Capecitabine, Cladribine, Clofarabine, Cytarabine, Fludarabine, and Gemcitabine. There are many nucleoside transporters and each may move these drugs across the membrane with unique kinetics.

A family of lipophilic gemcitabine analogs having an elaidic fatty acid esterified at the 5′ position has been produced. These analogs are able to transit the plasma membrane independent of the nucleoside transporters. The analogs are therefore believed to be more efficacious for patients who have tumors comprised of cancer cells with low levels of nucleoside transporters.

Accordingly, there exists a need for methods and therapies that identify patients that are likely to respond favorably to gemcitabine analogs rather than gemcitabine.

### SUMMARY OF THE INVENTION

The present invention provides methods and compositions for measuring the levels of nucleoside transporters in a tumor and correlating this level to a predicated efficacy of a given anti-cancer drug regime. The methods of the present invention allows for the treatment of cancer with a rationally selected and designed drug regime. In some aspects of the invention the level of hENT in cancer cells is determined and individuals with low levels of hENT 1 are treated with a lipophilic gemcitabine analog.

The invention provides methods for treating, delaying the progression of, preventing a relapse of, alleviating a symptom of, or otherwise ameliorating a cancer in a subject, e.g., a human subject, by detecting hENT1 expression level in the subject and comparing the hENT1 expression level in the subject with a control level of hENT1 expression level; and administering an effective dose of a gemcitabine analog such as, e.g., gemcitabine-5′-elaidate, in the range of 15 mg/kg to 100 mg/kg to ameliorate the cancer in the subject exhibiting a decreased level of hENT1 expression. For example, the gemcitabine analog is gemcitabine-5′-elaidate, and the gemcitabine-5′-elaidate is dosed in a range selected from 20 mg/kg to 100 mg/kg, 20 mg/kg to 90 mg/kg, 20 mg/kg to 80 mg/kg, 20 mg/kg to 70 mg/kg, 20 mg/kg to 60 mg/kg, 20 mg/kg to 50 mg/kg, 20 mg/kg to 40 mg/kg, 20 mg/kg to 30 mg/kg, 30 mg/kg to 100 mg/kg, 30 mg/kg to 90 mg/kg, 30 mg/kg to 80 mg/kg, 30 mg/kg to 70 mg/kg, 30 mg/kg to 60 mg/kg, 30 mg/kg to 50 mg/kg, 30 mg/kg to 40 mg/kg, 40 mg/kg to 100 mg/kg, 40 mg/kg to 90 mg/kg, 40 mg/kg to 80 mg/kg, 40 mg/kg to 70 mg/kg, 40 mg/kg to 60 mg/kg, 40 mg/kg to 50 mg/kg, 50 mg/kg to 100 mg/kg, 50 mg/kg to 90 mg/kg, 50 mg/kg to 80 mg/kg, 50 mg/kg to 70 mg/kg, 50 mg/kg to 60 mg/kg, 15 mg/kg to 50 mg/kg, 20 mg/kg to 50 mg/kg, 25 mg/kg to 50 mg/kg, 30 mg/kg to 50 mg/kg, 35 mg/kg to 50 mg/kg, 40 mg/kg to 50 mg/kg, 50 mg/kg to 95 mg/kg, 55 mg/kg to 90 mg/kg, 60 mg/kg to 90 mg/kg, 65 mg/kg to 90 mg/kg, 70 mg/kg to 90 mg/kg, 75 mg/kg to 90 mg/kg, 35 mg/kg to 75 mg/kg, 40 mg/kg to 75 mg/kg, 45 mg/kg to 75 mg/kg, 50 mg/kg to 75 mg/kg, 55 mg/kg to 75 mg/kg, and 60 mg/kg to 75 mg/kg.

For example, in some embodiments, the method for treating, delaying the progression of, preventing a relapse of, alleviating a symptom of, or otherwise ameliorating a cancer in a subject, e.g., a human subject, includes the steps of detecting hENT1 expression level in the subject and comparing the hENT1 expression level in the subject with a control level of hENT1 expression level; and administering an effective dose of gemcitabine-5′-elaidate in the range of 20 mg/kg to 80 mg/kg to ameliorate the cancer in the subject exhibiting a decreased level of hENT1 expression. For example, the gemcitabine analog is gemcitabine-5′-elaidate, and the gemcitabine-5′-elaidate is dosed in a range selected from 20 mg/kg to 80 mg/kg, 20 mg/kg to 70 mg/kg, 20 mg/kg to 60 mg/kg, 20 mg/kg to 50 mg/kg, 20 mg/kg to 40 mg/kg, 20 mg/kg to 30 mg/kg, 30 mg/kg to 80 mg/kg, 30 mg/kg to 70 mg/kg, 30 mg/kg to 60 mg/kg, 30 mg/kg to 50 mg/kg, 30 mg/kg to 40 mg/kg, 40 mg/kg to 80 mg/kg, 40 mg/kg to 70 mg/kg, 40 mg/kg to 60 mg/kg, 40 mg/kg to 50 mg/kg, 50 mg/kg to 80 mg/kg, 50 mg/kg to 70 mg/kg, 50 mg/kg to 60 mg/kg, 15 mg/kg to 50 mg/kg, 20 mg/kg to 50 mg/kg, 25 mg/kg to 50 mg/kg, 30 mg/kg to 50 mg/kg, 35 mg/kg to 50 mg/kg, 40 mg/kg to 50 mg/kg, 50 mg/kg to 80 mg/kg, 55 mg/kg to 80 mg/kg, 60 mg/kg to 80 mg/kg, 65 mg/kg to 80 mg/kg, 70 mg/kg to 80 mg/kg, 75 mg/kg to 80 mg/kg, 35 mg/kg to 75 mg/kg, 40 mg/kg to 75 mg/kg, 45 mg/kg to 75 mg/kg, 50 mg/kg to 75 mg/kg, 55 mg/kg to 75 mg/kg, and 60 mg/kg to 75 mg/kg.

In some embodiments, the control the control level of hENT1 expression has previously been determined from a source other than the subject. In some embodiments, the control level of hENT1 is contemporaneously determined from a source other than the subject. Suitable sources for these embodiments include any of those described herein.

In some embodiments, the control level is determined by obtaining a second non-cancerous sample from the subject. In some embodiments, the control level is determined by obtaining a non-cancerous sample from a different subject. Suitable samples include tissues, cells and biological fluids isolated from a subject, as well as tissues, cells and fluids present within a subject. Included within the usage of the term “biological sample” herein is blood and a fraction or component of blood including blood serum, blood plasma, or lymph.

In some embodiments, the control level is determined using the level of hENT1 expression in multiple control sources. Suitable control sources include any of those described herein.

In some embodiments, the control level is determined by obtaining a statistical distribution of hENT1 levels.

In some embodiments, the control level is determined from cultured cells engineered to express hENT1. In some embodiments, the control level is determined from cells engineered to not express hENT1. Suitable cell types include cells and cell lines recognized in the art as suitable for cell culture and/or any cells described herein.

In some embodiments, the control level is a clinically accepted reference level.

In some embodiments, the level of hENT1 expression in the subject is classified as high, medium or low according to an H-Score.

In some embodiments, the level of hENT1 expression in the subject is classified as a low sample when the H-Score is less than or equal to the overall median H-Score.

In some embodiments, effective dose of gemcitabine-5′-elaidate is administered as a single dose.

In some embodiments, the effective dose of gemcitabine-5′-elaidate is administered as multiple doses.

In some embodiments, the effective dose is administered every day, every third day, every third day times four, every third day times five, daily for ten consecutive days, or once weekly. For example, in some embodiments, the effective dose is administered every day as a single dose, every third day as a single dose, every third day times four as a single dose, every third day times five as a single dose, daily for ten consecutive days as a single dose, or once weekly as a single dose. For example, in some embodiments, the effective dose is administered every day as in multiple doses, every third day as in multiple doses, every third day times four as in multiple doses, every third day times five as in multiple doses, daily for ten consecutive days as in multiple doses, or once weekly as in multiple doses.

In some embodiments, the effective dose of gemcitabine-5′-elaidate is administered in a regimen selected from (i) doses of 25 mg/kg administered every third day, (ii) doses of 60 mg/kg administered every third day, (iii) doses of 80 mg/kg administered every third day, (iv) 4 mg/kg administered in a pattern of five consecutive days followed by two days off, (v) 40 mg/kg administered every third day times five, (vi) 40 mg/kg administered weekly, (vii) 40 mg/kg or 150 mg/kg administered once weekly times two or administered every third day times five, (viii) 75 mg/kg/dose administered every third day times four, (ix) 5 mg/kg/dose administered daily times five, (x) 1 mg/kg, 4 mg/kg or 75 mg/kg administered as a single dose or administered daily for ten consecutive days, (xi) 80 mg/kg administered intraperitoneally, (xii) 7.5, 15, 20, 22.5, 30 or 40 mg/kg administered every three days times five, daily times five or once weekly times two, orally, and any combinations thereof.

In some embodiments, the effective dose is administered intravenously, subcutaneously, orally or a combination thereof

In some embodiments, the level of hENT1 expression in the subject as compared to the level of expression of hENT1 in the control subject is decreased by at least one-fold, two-fold, five-fold, ten-fold or more. In some embodiments, the level of hENT1 expression in the subject as compared to the level of expression of hENT1 in the control subject is decreased by at least 1, % 5%, 10%, 15%, 25%, 30%, 35%, 40%, 45%, 50%, 55%, 60%, 65%, 70%, 75%, 80%, 85%, 90%, 95%, or more.

The terms “subject” and “patient” are used interchangeably herein. Preferably, the subject is human.

In some embodiments, the subject is non-responsive, less responsive or has stopped responding to treatment with a chemotherapeutic agent. For example, in some embodiments, the chemotherapeutic agent is gemcitabine. Other suitable chemotherapeutic agents include those recognized in the art and/or any of the chemotherapeutic and anti-neoplastic agents described herein.

In some embodiments, the cancer is renal cancer, including renal cell carcinoma, glioblastoma, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas including Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, environmentally induced cancers including those induced by asbestos, e.g., mesothelioma or a combinations of said cancers.

In some embodiments, the cancer is non-small cell lung cancer (NSCLC), sarcoma, malignant melanoma, prostate cancer, breast cancer, pancreatic cancer, colon cancer including a colon carcinoma, glioma, leukemia, or liver cancer.

In some embodiments, the gemcitabine-5′-elaidate is administered at an effective dose in the range of 15 mg/kg to 50 mg/kg. In some embodiments, the gemcitabine-5′-elaidate is administered at an effective dose in a range selected from 20 mg/kg to 50 mg/kg, 20 mg/kg to 40 mg/kg, 20 mg/kg to 30 mg/kg, 30 mg/kg to 50 mg/kg, 30 mg/kg to 40 mg/kg, 40 mg/kg to 50 mg/kg, 15 mg/kg to 50 mg/kg, 20 mg/kg to 50 mg/kg, 25 mg/kg to 50 mg/kg, 30 mg/kg to 50 mg/kg, 35 mg/kg to 50 mg/kg, and 40 mg/kg to 50 mg/kg.

The invention provides methods for treating, delaying the progression of, preventing a relapse of, alleviating a symptom of, or otherwise ameliorating a cell proliferation disease or other disorder in a human, mammal, or animal subject afflicted with that disease or disorder.

Administration of the gemcitabine analogs, e.g., gemcitabine-5′-elaidate, and/or pharmaceutical compositions thereof to a patient suffering from a cell proliferation disease or disorder is considered successful if any of a variety of laboratory or clinical results is achieved. For example, administration is considered successful one or more of the symptoms associated with the cell proliferation disease or disorder is alleviated, reduced, inhibited or does not progress to a further, i.e., worse, state. Administration is considered successful if the cell proliferation disorder, e.g., cancer or other neoplastic condition, enters remission and/or does not progress to a further, i.e., worse, state.

In some embodiments, the gemcitabine analogs, e.g., gemcitabine-5′-elaidate, and/or pharmaceutical compositions thereof are administered in combination with any of a variety of known therapeutics, including for example, chemotherapeutic and other anti-neoplastic agents, anti-inflammatory compounds and/or immunosuppressive compounds. In some embodiments, the gemcitabine analogs, e.g., gemcitabine-5′-elaidate, and/or pharmaceutical compositions thereof are useful in conjunction with any of a variety of known treatments including, by way of non-limiting example, surgical treatments and methods, radiation therapy, chemotherapy and/or hormone or other endocrine-related treatment.

In some embodiments, the gemcitabine-5′-elaidate is administered in combination with one or more chemotherapeutic and/or cytotoxic agents. Suitable agents and/or cytotoxic agents include those recognized in the art and/or any of the chemotherapeutic agents, anti-neoplastic agents and/or cytotoxic agents described herein.

These “co-therapies” can be administered sequentially or concurrently. The gemcitabine analogs, e.g., gemcitabine-5′-elaidate, and/or pharmaceutical compositions thereof and the additional agent(s) can be administered to a subject, preferably a human subject, in the same pharmaceutical composition. Alternatively, the gemcitabine analogs, e.g., gemcitabine-5′-elaidate, and/or pharmaceutical compositions thereof and the second agent(s) can be administered concurrently, separately or sequentially to a subject in separate pharmaceutical compositions. The gemcitabine analogs, e.g., gemcitabine-5′-elaidate, qnd/or pharmaceutical compositions thereof and the second therapy may be administered to a subject by the same or different routes of administration.

In some embodiments, the co-therapies of the invention comprise an effective amount of the gemcitabine analogs, e.g., gemcitabine-5′-elaidate, and/or pharmaceutical compositions thereof and an effective amount of at least one other therapy (e.g., prophylactic or therapeutic agent) which has a different mechanism of action than the gemcitabine analogs described herein, e.g., gemcitabine-5′-elaidate. In some embodiments, the co-therapies of the present invention improve the prophylactic or therapeutic effect of the gemcitabine analogs, e.g., gemcitabine-5′-elaidate, and of the second therapy by functioning together to have an additive or synergistic effect. In some embodiments, the co-therapies of the present invention reduce the side effects associated with the second therapy (e.g., prophylactic or therapeutic agents).

In some embodiments, the gemcitabine analogs, e.g., gemcitabine-5′-elaidate, are administered in combination with the additional agent(s). The term “in combination” in this context means that the gemcitabine analogs, e.g., gemcitabine-5′-elaidate, and the additional agent(s) are given substantially contemporaneously, either simultaneously or sequentially. If given sequentially, at the onset of administration of the second compound, the first of the two compounds is preferably still detectable at effective concentrations at the site of treatment. The gemcitabine analog, e.g., gemcitabine-5′-elaidate, can be administered first and the additional agent(s) can be administered second, or alternatively, the additional agent(s) can be administered first and the gemcitabine analog, e.g., gemcitabine-5′-elaidate, can be administered second.

In some embodiments, combination therapy can include one or more gemcitabine analogs, e.g., gemcitabine-5′-elaidate, coformulated with one or more additional agents.

The gemcitabine analogs, e.g., gemcitabine-5′-elaidate, and additional agent(s) can be administered by the same or by different routes of administration.

In some embodiments, the disease or disorder can be treated by administering the compound, product and/or pharmaceutical composition as follows. The blood molar concentration of the compound can be at least an effective concentration and less than a harmful concentration for a first continuous time period that is at least as long as an effective time period and shorter than a harmful time period. The blood molar concentration can be less than the effective concentration after the first continuous time period. For example, the effective time period can be about 1 hour, 2 hour, about 4 hours, about 6 hours, about 8 hours, about 10 hours, about 12 hours, about 24 hours, or another time period determined to be effective by one of skill in the art. For example, the harmful time period can be about 12 hours, about 24 hours, about 48 hours, about 72 hours, about 144 hours, or another time period determined to be harmful by one of skill in the art.

In some embodiments, the therapeutically effective amount of the compound, product and/or pharmaceutical composition is selected to produce a blood concentration greater than the IC₅₀ of cells of the tumor and less than the IC₅₀ of normal cells. In some embodiments, the therapeutically effective amount is selected to produce a blood concentration sufficiently high to kill cells of the tumor and less than the IC₅₀ of normal cells.

In some embodiments, the compound, product and/or pharmaceutical composition is administered orally in a dosage form, for example, a tablet, pill, capsule (hard or soft), caplet, powder, granule, suspension, solution, gel, cachet, troche, lozenge, syrup, elixir, emulsion, oil-in-water emulsion, water-in-oil emulsion, and/or a draught.

The invention provides methods for treating, delaying the progression of, preventing a relapse of, alleviating a symptom of, or otherwise ameliorating a cancer in a subject, e.g., a human subject, by detecting hENT1 expression level in the subject and comparing the hENT1 expression level in the subject with a control level of hENT1 expression level; and administering an effective dose of a gemcitabine analog such as, e.g., gemcitabine-5′-elaidate, to ameliorate the cancer in the subject exhibiting a decreased level of hENT1 expression, wherein the effective dose of gemcitabine-5′-elaidate is administered in a regimen selected from (i) doses of 25 mg/kg administered every third day, (ii) doses of 60 mg/kg administered every third day, (iii) doses of 80 mg/kg administered every third day, (iv) 4 mg/kg administered in a pattern of five consecutive days followed by two days off, (v) 40 mg/kg administered every third day times five, (vi) 40 mg/kg administered weekly, (vii) 40 mg/kg or 150 mg/kg administered once weekly times two or administered every third day times five, (viii) 75 mg/kg/dose administered every third day times four, (ix) 5 mg/kg/dose administered daily times five, (x) 1 mg/kg, 4 mg/kg or 75 mg/kg administered as a single dose or administered daily for ten consecutive days, (xi) 80 mg/kg administered intraperitoneally, (xii) 7.5, 15, 20, 22.5, 30 or 40 mg/kg administered every three days times five, daily times five or once weekly times two, orally, and any combinations thereof

In one aspect, the invention provides for a method for treating cancer in an individual by determining the level of nucleoside transporter in an sample derived from an individual in need of the treatment of a cancer, and transmitting data pertaining to the nucleoside transporter level to a physician who provides an instruction regarding administering a therapeutically effective amount of a chemotherapeutic nucleoside analog to the individual based on the nucleoside transporter level.

In one aspect of the invention the amount of the chemotherapeutic nucleoside analog is determined based upon the level of nucleoside transporter.

In one aspect of the invention a particular chemotherapeutic nucleoside analog is administered based upon the level of nucleoside transporter.

In one aspect of the invention the cancer is metastatic pancreatic cancer.

In one aspect of the invention the sample is a biopsy containing cancer cells.

In one aspect of the invention the biopsy is fine needle aspiration of pancreatic cancer cells.

In one aspect of the invention the biopsy is laparoscopy obtained pancreatic cancer cells.

In one aspect of the invention the biopsied cells are centrifuged into a pellet, fixed, and embedded in paraffin.

In one aspect of the invention the biopsied cells are flash frozen.

In one aspect of the invention the biopsied cells are mixed with an antibody that recognizes the nucleoside transporter.

In one aspect of the invention the sample comprises a circulating metastatic pancreatic cancer cell.

In one aspect of the invention the sample is obtained by sorting pancreatic CTCs from blood.

In one aspect of the invention the nucleoside transporter is hENT1, hENT2, hENT3, hENT4, hCNT1, hCNT2, or hCNT3.

In one aspect of the invention the nucleoside transporter is hENT1.

In one aspect of the invention the hENT1 level is determined by hENT1 antibody staining.

In one aspect of the invention the antibody staining is performed for less than 12 hours.

In one aspect of the invention the antibody staining is performed for more than 12 hour.

In one aspect of the invention the hENT1 level is determined by mRNA level.

In one aspect of the invention the level of nucleoside transporter is a measure of the ability of hydrophilic chemotherapeutic nucleoside analogs to enter a cancer cell.

In one aspect of the invention the nucleoside transporter level is a determined by SNP analysis.

In one aspect of the invention the nucleoside transporter level is a determined by the identification of a polymorphism.

In one aspect of the invention a control level of nucleoside transporter has previously been determined from a source other than the sample.

In one aspect of the invention a control level of nucleoside transporter is contemporaneously determined from a source other than the sample.

In one aspect of the invention the control level is determined by obtaining a second non-cancerous sample from the individual.

In one aspect of the invention the control level is determined by obtaining a non-cancerous sample from a different individual.

In one aspect of the invention the control level is determined by obtaining statistical distribution of nucleoside transporter levels.

In one aspect of the invention the control level is determined from cultured cells engineered to express hENT1.

In one aspect of the invention the control level is determined from cells engineered to not express hENT1.

In one aspect of the invention the sample is classified as a low nucleoside transporter level sample when more than 50% of the cells do not have strong reactivity for antibody staining.

In one aspect of the invention the nucleoside transporter is hENT1 and the antibody is a hENT1 antibody.

In one aspect of the invention the sample is classified as a low nucleoside transporter sample when the percentage of the sample with no nucleoside transporter staining is greater than the overall median percentage of stained cells.

In one aspect of the invention the nucleoside transporter is hENT1 and the antibody is a hENT1 antibody.

In one aspect of the invention the sample is classified according to an H-Score.

In one aspect of the invention the sample is classified as a low nucleoside transporter sample when the H-Score is less than or equal to the overall median H-Score.

In one aspect of the invention the nucleoside transporter is hENT1 and the antibody is a hENT1 antibody.

In one aspect of the invention the sample is classified as a low hENT1 sample when more than 50% of the cells do not have strong reactivity for hENT1 antibody staining.

In one aspect of the invention the sample is classified as a low hENT1 sample when the mRNA level of the sample is less than 5%, 10%, 20%, 30%, 40%, 50%, 60%, 70% or 80% of the mRNA level of the control.

In one aspect of the invention the chemotherapeutic nucleoside analog is Capecitabine, Cladribine, Clofarabine, Cytarabine, Fludarabine, or Gemcitabine.

In one aspect of the invention the chemotherapeutic nucleoside analog is a hydrophobic derivative of Capecitabine, Cladribine, Clofarabine, Cytarabine, Fludarabine, or Gemcitabine.

In one aspect of the invention the chemotherapeutic nucleoside analog is gemcitabine-5′-elaidate.

In one aspect of the invention the gemcitabine-5′-elaidate is administered subcutaneously in a dose of 25-80 mg/kg every third day.

In one aspect the invention provides for a method for treating metastatic pancreatic cancer in an individual by administering gemcitabine-5′-elaidate to an individual in need of the treatment of a cancer; and administering a nucleoside transporter inhibitor to the individual, wherein the inhibitor prevents efflux of the gemcitabine-5′-elaidate from cancer cells.

In one aspect of the invention the nucleoside transporter inhibitor is dipyridamole or NBMPR.

In one aspect of the invention the treatment of metastatic pancreatic cancer, comprises administering gemcitabine-5′-elaidate to a subject in need of the treatment of a cancer, wherein the hydrophobic tail of gemcitabine-5′-elaidate is cleaved by an extracellular esterase resulting in gemcitabine.

In one aspect of the invention the gemcitabine-5′-elaidate is co-administered with gemcitabine.

In one aspect the invention provides a method for determining high or low expression of hENT1 by generating hENT1 positive cells, generating hENT1 negative cells, and comparing the binding of an antibody that recognizes hENT1 on the positive and negative cells.

In one aspect of the invention the negative cells are HeLa knock down cells, or CEN negative control

In one aspect of the invention the expression level is the protein expression level of the transporter.

In one aspect of the invention the expression level is the mRNA expression level of the transporter.

In one aspect of the invention the transporter is hCNT1.

In one aspect of the invention the individual is a human.

In one aspect of the invention the chemotherapeutic nucleoside analog is a lipid-conjugated gemcitabine.

In one aspect of the invention the determination step comprises comparing the expression level of the transporter in the sample to the expression level of the transporter in a control sample.

In one aspect of the invention the method further comprises directing the administration of a nucleoside transporter blocker to the individual.

In one aspect of the invention the transporter is administered to the individual in about 2 hours after the administration of the gemcitabine analog.

In one aspect the invention provides a kit comprising an agent for the determination of hENT1 expression level.

In one aspect of the invention the kit further comprises a control sample.

In one aspect of the invention the kit further comprises instructions for classifying a hENT1 expression level in a sample.

In one aspect of the invention the instructions are provided electronically to purchasers of the kit.

In one aspect of the invention he kit further comprises gemcitabine-5′-elaidate.

In one aspect of the invention the method further comprising making gemcitabine-5′-elaidate available to the individual.

In one aspect the invention provides a method of directing treatment of a disease by delivering a sample suspected of having a low level of functional hENT 1 to a diagnostic lab for determination of hENT1 levels; providing a control sample with a known level of hENT; providing an antibody to hENT1; subjecting the sample and control sample to binding by the antibody, and detecting a relative amount of antibody binding, wherein a sample with a low amount of hENT1 binding is used to provide a conclusion that a patient should receive gemcitabine-5′-elaidate.

In one aspect of the invention the method of directing treatment of a disease further comprises reviewing or analyzing data relating to the presence of hENT1 in a sample; and providing a conclusion to an individual, a health care provider or a health care manager, the conclusion being based on the review or analysis of data. In one aspect of the invention a conclusion is the transmission of the data over a network.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 illustrates the various aspects of the invention. A patient with a tumor 101 is identified. A device 103 is used to extract a sample 105 from the patient. The sample is analyzed in an analyzer 107 to determine the status of one or more transporters.

The results of the analysis are used to determine which, if any, therapeutic 109 should be provided to the patient to treat a tumor.

FIG. 2 depicts specific nucleoside transporters through which hydrophilic nucleosides can transit a lipid membrane.

FIG. 3 illustrates that gemcitabine-5′-elaidate enters tumor cells despite blocking of nucleoside transporters. Gemcitabine triphosphate (dFdCTP) accumulates in cells despite blocking transporters post gemcitabine-5′-elaidate administration.

FIG. 4 depicts that blocking transporters does not protect against gemcitabine-5′-elaidate cell killing in vitro. The number on the Y axis is the fold protection by dipyridamole from administration of gemcitabine and gemcitabine-5′-elaidate. This effect was demonstrated in four cell lines: THX, Lox, MOLT4, and MOLT4/C.

FIGS. 5A-5B depict that gemcitabine-5′-elaidate kills tumor cells in vitro independent of hENT1. CEM: T-lymphoblastic leukemia cell line. CEM-Ara-C/8: Clonal derivative of CEM with hENT1 transporter deficiency.

FIG. 6 depicts the correlation of hENT1 mRNA levels and sensitivity to gemcitabine in xenograft mouse models.

FIG. 7 is a block diagram showing a representative example logic device through which reviewing or analyzing data relating to the present invention can be achieved.

FIG. 8 depicts a hypothetical Kaplan-Meier plot of survival for patients receiving gemcitabine monotherapy -Patients with homogeneous hENT1 expression (dotted line) outperformed those with areas of hENT1-low tumor.

### DETAILED DESCRIPTION

### I. Introduction

In one aspect, the present invention provides a method for treating a patient with cancer. The method comprises predicting an individual's response to a cancer therapy, including a response by human cancer patients. Methods for matching a particular chemotherapeutic agent to particular individual based on predicted efficacy, and methods for directing treatment and informing patients and physicians are also provided.

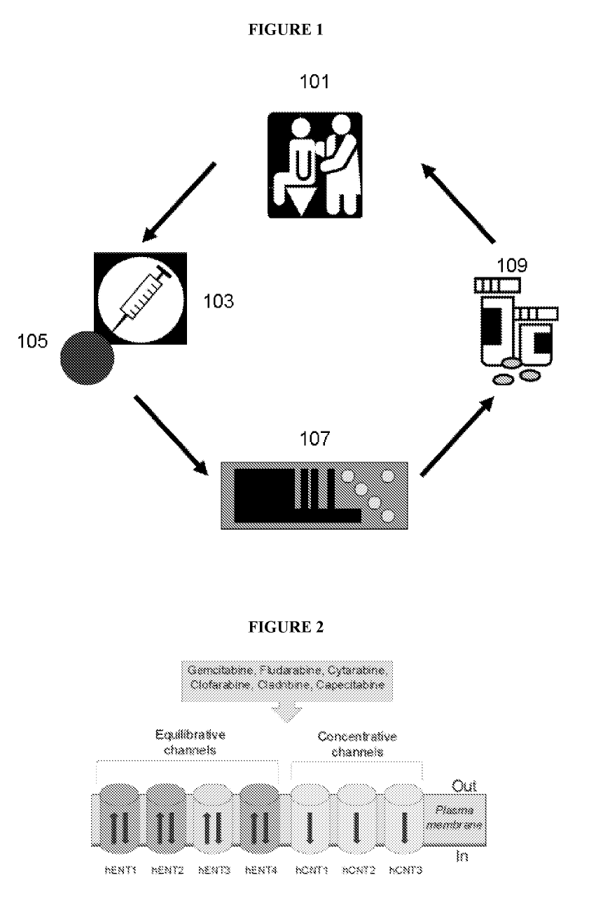

In order to treat a cancer patient, the present invention teaches the following general aspects. First, a cancer patient is identified. A sample of this individual's tumor is obtained and analyzed to determine the level of expression of one or more nucleoside transporters. This information is then used to determine which of the available anticancer drugs the patient should use. For instance, a patient that lacks or has low levels of nucleoside transporters is informed that hydrophilic nucleoside anticancer drugs are not likely to be efficacious as these drugs are not likely to enter the cancer cells. Such a patient can be given derivatives of these drugs which have been modified to enter the cancer cells independent of the transporters. An exemplary embodiment of the invention is depicted in FIG. 1. A patient with a tumor 101 is identified. A device 103 is used to extract a sample 105 from the patient. The sample is analyzed in an analyzer 107 to determine the status of one or more transporters. The results of the analysis are used to determine which, if any, therapeutic 109 should be provided to the patient to treat a tumor.

### II. Identification of Individuals

The identification of individuals for the practice of the invention is preformed using any known diagnostic technique. The identification can be performed by a physician. The identification of the individual can be by communication with the physician, the individual, a health care company, an insurer, or from a computer database which stores data related to the individual. In some embodiments, the identification of the individual is concurrent with the testing of the samples. In some embodiments, the individual is identified and then further testing is performed.

In some embodiments, the term “individual” is synonymous with “a patient” or “a subject”. In some embodiments, the individual is suspected of having cancer. In some embodiments, the individual has been diagnosed with cancer. In some embodiments, the individual has been proven to have cancer.

In some embodiments, the individual is a human, however in some embodiments the individual is a non-human mammal In some embodiments, the non-human mammal is a domesticated animal with cancer.

In some embodiments, methods described herein are useful for ameliorating a cancer in an individual. Amelioration includes, but is not limited to, treating, suppressing and/or preventing a cancer in a subject.

Methods described herein are related to a variety of cancers. In some instances, cancer can be a metastatic cancer. Examples of cancers related to the methods described herein include, but are not limited to, non-small cell lung cancer (NSCLC), sarcoma, malignant melanoma, prostate cancer, breast cancer, pancreatic cancer, colon cancer (such as a colon carcinoma), glioma, leukemia, liver cancer, colon cancer (including small intestine cancer), lung cancer, breast cancer, pancreatic cancer, melanoma (e.g., metastatic malignant melanoma), acute myeloid leukemia, kidney cancer, bladder cancer, ovarian cancer, prostate cancer, renal cancer (e.g., renal cell carcinoma), glioblastoma, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas (e.g., Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, or environmentally induced cancers including those induced by asbestos, e.g., mesothelioma. In some embodiments, methods described herein can be useful for treating a combination of two or more types of cancer. In some embodiments, the methods are useful to treat individual patients diagnosed with cancer.

In some embodiments, the individual is in the midst of an ongoing therapeutic regime. In some embodiments, the individual has not yet received treatment. In some embodiments, the individual is subjected to a diagnostic test in the midst of an ongoing therapeutic regime so as to identify levels of transporters such as hENT1 or hCNT1 in cancerous cells or tissue.

In some embodiments, the individual has a disorder other than cancer that is treated with hydrophilic nucleoside drugs.

### III. Obtaining Samples

Generally, to perform a diagnostic test, a sample is obtained from a patient in need of therapy. In the present invention, once the individual is identified the present invention provides methods for obtaining a sample from the individual. In some aspects the sample is a tumor sample. In some aspects the sample contains cancer cells. In some aspects the sample contains cells which are likely to correlate to the transporter levels in the individual's cancer cells.

In some embodiments, the nucleoside transporter level is determined in a sample from a tissue, organ, cell, and/or tumor. In some embodiments, the nucleoside transporter level is determined in a circulating tumor cell. In some embodiments, the nucleoside transporter level is determined in a biopsy sample.

### A. Circulating Tumor Cells (CTCs)

In some embodiments, the nucleoside transporter level is determined in a sample of a bodily fluid. In some embodiments, a bodily fluid sample is taken from an individual and nucleoside transporter levels are obtained from the bodily fluid. In some embodiments, the levels of nucleoside transporter are obtained from a subset of cells obtained in a bodily fluid sample.

Bodily fluids include but are not limited to blood, lymph, saliva, semen, CSF, breast milk, peritoneal fluid, and pleural effusion.

In some embodiments, a blood sample is taken and nucleoside transporter levels are obtained. In some embodiments, the levels of nucleoside transporter are obtained from a subset of cells obtained in a blood sample.

In some embodiments, a lymph sample is taken from a subject and nucleoside transporter levels are obtained from the lymph. In some embodiments, the levels of nucleoside transporter are obtained from a subset of cells obtained in a lymph sample.

In some embodiments, a saliva sample is taken from a subject and nucleoside transporter levels are obtained from the saliva. In some embodiments, the levels of nucleoside transporter are obtained from a subset of cells obtained in a saliva sample.

In some embodiments, a semen sample is taken from a subject and nucleoside transporter levels are obtained from the semen. In some embodiments, the levels of nucleoside transporter are obtained from a subset of cells obtained in a semen sample.

In some embodiments, a cerebrospinal fluid (CSF) sample is taken from a subject and nucleoside transporter levels are obtained from the CSF. In some embodiments, the levels of nucleoside transporter are obtained from a subset of cells obtained in a CSF sample.

In some embodiments, a breast milk sample is taken from a subject and nucleoside transporter levels are obtained from the breast milk. In some embodiments, the levels of nucleoside transporter are obtained from a subset of cells obtained in a breast milk sample.

In some embodiments, a peritoneal fluid sample is taken from a subject and nucleoside transporter levels are obtained from the peritoneal fluid. In some embodiments, the levels of nucleoside transporter are obtained from a subset of cells obtained in a peritoneal fluid sample.

In some embodiments, a pleural effusion sample is taken from a subject and nucleoside transporter levels are obtained from the pleural effusion. In some embodiments, the levels of nucleoside transporter are obtained from a subset of cells obtained in a pleural effusion sample.

In some embodiments, the nucleoside transporter level is determined in a circulating tumor cell CTCs are cells that have detached from a primary tumor and circulate in a bodily fluid. In some embodiments, the nucleoside transporter level is determined in a circulating tumor cell CTCs are cells that have detached from a primary tumor and circulate in the bloodstream. CTCs may constitute seeds for subsequent growth of additional tumors (metastasis) in different tissues.

In certain embodiments the CTCs are collected using methods described in U.S. Pat. Nos. 5,466,574; 5,512,332; 5,597,531; 5,698,271; 5,985,153; 5,993,665; 6,120,856; 6,136,182; 6,365,362; 6,551,843; 6,620,627; 6,623,982; 6,645,731; 6,660,159; 6,790,366; 6,861,259; 6,890,426; 7,011,794, 7,282,350, 7,332,288, 5,849,517 and 5,459,073.

### B. Biopsy

In some embodiments, the nucleoside transporter level is determined in a biopsy sample. In some embodiments, the sample is fine needle aspiration tumor cells. In some embodiments, the sample is laparoscopy obtained tumor cells. In some embodiments, the sample is surgically obtained tumor cells. In some embodiments, a biopsy is taken to determine whether an individual has cancer and is then later used as a sample for the present invention.

In some embodiments, the nucleoside transporter level is determined in a biopsy sample. In some embodiments, the sample is fine needle aspiration of pancreatic cancer cells. In some embodiments, the sample is laparoscopy obtained pancreatic cancer cells.

In some embodiments, the samples are formalin fixed biopsy samples. In some embodiments, the samples are frozen. In some embodiments, the samples have been stored for a period of time.

### C. Control Samples

In some embodiments, the level of expression of nucleoside transporter in a subject is compared to the level of expression of nucleoside transporter in a control sample. In some embodiment the level of expression of nucleoside transporter in a subject is compared to the level of expression of nucleoside transporter in multiple control samples. In some embodiments, multiple control samples are used to generate a statistic that is used to classify the level of expression of nucleoside transporter in an individual with cancer.

Control samples can be obtained using the same sources and methods as non-control samples.

In some embodiments, the control sample is obtained from a person other than the patient. In some embodiments, multiple control samples are obtained from multiple people. In some embodiments, the other people are relatives of the patient. In some embodiments, the other people are from a genetically similar group as the patient, e.g., ashkanazi.

In some embodiments, the level of expression of nucleoside transporter in a sample derived from a subject is compared to the level of expression of nucleoside transporter in non-cancerous cells from another individual. In some embodiments, the level of expression of nucleoside transporter in a sample derived from an individual's cancer cells is compared to the level of expression of nucleoside transporter in non-cancerous cells from another individual. In some embodiments, the level of expression of nucleoside transporter in a sample from an individual is compared to the level of expression of nucleoside transporter in cancerous cells from another individual.

In some embodiments, the control sample is tissue, cells, or organs obtained from a healthy individual.

In some embodiments, the control sample is tissue, cells, or organs obtained from an individual with cancer.

In some embodiments, multiple control samples are used to determine a range of expression of nucleoside transporter in a particular tissue, organ, or cell population. In some embodiments, the multiple control samples are from many different individuals. In some embodiments, the many individuals from whom the control samples are taken do not have cancer. In some embodiments, the many individuals from whom the control samples are taken have cancer. In some embodiments, some of the many individuals from whom the control samples are taken have cancer and some individuals do not, thereby providing both positive and negative control samples.

In some instances control samples are samples that were obtained from other patients for testing. In some instances control samples were not collected with the intention of using them as control samples.

In some instances control samples are collected post mortem.

In some embodiments, the control sample is from a patient in need of treatment. In some embodiments, the control sample is a normal tissue from the same patient that in need of treatment.

In some embodiments, the control sample is a cultured tissue or cell that has been determined to be a proper control.

In some embodiments, the control is a cell that does not express one or more transporter genes (negative control). Such cells are obtained by knock out or knock down the endogenous transporter genes in the cells by methods known in the art, such as by RNAi. In some embodiments, the negative control cell is a HeLa cell wherein the expression of a transporter gene, such as hENT1 and/or hCNT1, is knocked out or knocked down.

In some embodiments, the control cell is a cultured cell derived from an individual. In some embodiments, the control cell is a cultured cell derived from a patient.

In some embodiments, the control is a cell that expresses one or more transporter genes. Most cells express transporter genes, and thus can be used as the positive control.

In some embodiments, a reference level clinically accepted as normal level in a standardized test can be used to determine the level of expression in tumor sample. In some embodiments, determination of high or low expression level is made in reference to a control sample obtained from a control subject other than the patient. A control subject can be a person who is deemed to be a healthy person sharing similar ethnic, age, and gender identity with the patient.

In some embodiments, the nucleoside transporter level determined in the control sample is the level of hENT1, hENT2, hENT3, hENT4, hCNT1, hCNT2, or hCNT3. In some embodiments, multiple nucleoside transporter levels are determined in the control sample. The levels of all or a subset of the nucleoside transporter levels can be compared to a sample from an individual with cancer.

In some embodiments, the nucleoside transporter level determined in the control sample or control samples is hENT1.

### IV. Storage and Transport of Samples

In some aspects samples may be obtained at a different location from where the determining of nucleoside transporter levels occurs. In such embodiments the samples are transported. In some embodiments, samples may be obtained at different times than when the determining of nucleoside transporter levels occurs. In such embodiments the samples are stored.

In some embodiments, control samples are shipped, sold, or transported prior to comparison with a sample from a patient. In some embodiments, samples from cancer patients are shipped, sold, or transported prior to comparison with control cells.

In some embodiments, the biological samples are fixed samples, e.g., a formalin fixed, paraffin-embedded (FFPE) sample, or a frozen samples.

In some embodiments, the only portions of the samples are shipped. Nonlimiting examples of shipped portions are sorted cancer cells or isolated lipid membranes.

### V. Nucleoside Transporters

In some aspects the present invention involves the detection of nucleoside transporters. In some embodiments, the levels of particular nucleoside transporters are determined In some embodiments, the levels of multiple nucleoside transporters are determined In some embodiments, the level of hENT1 is determined.

Nucleoside transporters are a group of membrane transport proteins. Nucleoside transporters move physiologic nucleosides through the plasma membrane. Nucleoside transporters transport nucleoside substrates such as adenosine across the membranes of cells and/or vesicles. Nucleoside transporters can also move anticancer and antiviral nucleoside drugs across the plasma membrane.

The two major classes of nucleoside transporters in human cells and tissues are equilibrative nucleoside transporters (ENTs) and concentrative nucleoside transporters (CNTs).

The equilibrative nucleoside transporter (ENT) family, also known as SLC29, is a group of plasmalemmnal transport proteins which transport nucleoside substrates such as adenosine into cells. There are four known ENTs, designated ENT1, ENT2, ENT3, and ENT4. ENTs are blocked by adenosine reuptake inhibitors such as dipyridamole and dilazep.

The concentrative nucleoside transporter (CNT) family, also known as SLC28, has three members: SLC28A1, SLC28A2 and SLC28A3, also designated as CNT1, CNT2 and CNT3.

FIG. 2 depicts some nucleoside transporters through which hydrophilic nucleosides can enter cells. These transporters include hENT1, hENT2, hENT3, hENT4, hCNT1, hCNT2, and hCNT3. The arrows indicate that some transporters can move hydrophilic nucleosides in both directions across the plasma membrane.

There is variability in the ability for the different nucleoside transporters to move particular compounds across the plasma membrane. For instance hENT2 transports hypoxanthine and other purine nucleobases better than hENT1. hENT1 transports gemcitabine more efficiently than other known nucleoside transporters.

Human equilibrative nucleoside transporter 1 (hENT1) is a protein that is encoded by the SLC29A1 gene. This gene is a member of the equilibrative nucleoside transporter family. The gene encodes a transmembrane glycoprotein that localizes to the plasma and mitochondrial membranes and mediates the cellular uptake of nucleosides from the surrounding medium. The protein is categorized as an equilibrative (as opposed to concentrative) transporter that is sensitive to inhibition by nitrobenzylthioinosine (NBMPR). Nucleoside transporters are required for nucleotide synthesis in cells that lack de novo nucleoside synthesis pathways, and are also necessary for the uptake of cytotoxic nucleosides and nucleoside analog drugs used for cancer and viral chemotherapies.

hCNT1 (solute carrier family 28 (sodium-coupled nucleoside transporter), member 1), is disclosed in U.S. Pat. No. 6,153,740.

VI. Assays for the Determination of Transporter Expression Level

In yet another aspect the present invention provides for methods for determining the level of one or more nucleoside transporters in a sample. In some aspects a “level” is the activity level of a nucleoside transporter in a sample, wherein the activity level is a measure of the total amount of hydrophilic nucleosides that are moved across the membrane by the nucleoside transporter in a cell, a sample, or a tumor. In some aspects the level is an expression level that correlates to the activity level. In some aspects the expression level is a measure of a protein present in a cell, a sample, or a tumor. In some aspects the expression level is a measure of a nucleic acid present in a cell, a sample, or a tumor.

In some aspects the present invention provides methods for the determination of the expression level of a biomarker. The biomarker is indicative of the level of nucleoside transporter expression in a sample or control sample. The expression level of the biomarker is determined by performing one or of the assays provided herein and known in the art on a sample obtained from the subject that is either has a cancer condition or is a healthy individual. Control samples can be assayed using the same methods as non-control samples.

In some embodiments, the biomarker is a nucleoside transporter protein. In some embodiments, the biomarker is DNA encoding a nucleoside transporter. In some embodiments, the biomarker is mRNA encoding a nucleoside transporter. In some embodiments, the biomarker is mRNA encoding a protein which interacts with a nucleoside transporter. In some embodiments, the biomarker is a nucleic acid related to nucleoside transporter expression levels. In some embodiments, the biomarker is a SNP related to expression levels of a nucleoside transporter.

In some embodiments, the biomarker is a nucleic acid related to a mutation which alters the ability of a nucleoside transporter to move a chemotherapeutic agent across a plasma membrane.

In some embodiments, the present invention provides biomarkers to determine the level of transporters in a patient. In some embodiments, the present invention provides for methods of determining the level of the transporter related biomarker in a sample from an individual useful for determining whether administering a chemotherapeutic agent will be effective. In some embodiments, the present invention provides methods for determining the level of the transporter related biomarker in a sample from an individual useful for determining which chemotherapeutic agent should be used.

In some aspects, the expression of the transporters in relevant cells, organs, tumors or tissues differs between individuals. In some aspects, the expression differences include lack of expression, increase expression or expression or a region that is polymorphic in a population. In some aspects the expression differences are based upon genomic differences between individuals or based upon regulation of gene expression in a particular individual, cell, organ, tumor or tissue at a particular time.

### A. Nucleic Acids

Gene expression can be evaluated using mRNA extracted from a sample using standard methods in the art. mRNA analysis methods include but are not limited to hybridization methods, such as Northern blot analysis, and amplification methods, such as real-time PCR. A high throughput platform can be used to process two or more samples.

In some embodiments, the mRNA is detected using a nucleic acid probe. By a nucleic acid probe is meant to include a collection of one or more nucleic acid fragments whose hybridization to a sample can be detected. The probe can be unlabeled or labeled so that its binding to the target or sample can be detected. A probe can be produced from a source of nucleic acids from one or more particular (preselected) portions of the genome, e.g., one or more clones, an isolated whole chromosome or chromosome fragment, or a collection of polymerase chain reaction (PCR) amplification products. The nucleic acid probe can also be isolated nucleic acids immobilized on a solid surface (e.g., nitrocellulose, glass, quartz, fused silica slides), as in an array. The probe can be a member of an array of nucleic acids. Techniques capable of producing high density arrays can also be used for this purpose (see, e.g., Fodor (1991) Science 767-773; Johnston (1998) Curr. Biol. 8: R171-R174; Schummer (1997) Biotechniques 23: 1087-1092; Kern (1997) Biotechniques 23: 120-124; U.S. Pat. No. 5,143,854). One of skill in the art will recognize that the precise sequence of the particular probes can be modified to a certain degree to produce probes that are “substantially identical,” but retain the ability to specifically bind to (i.e., hybridize specifically to) the same targets or samples as the probe from which they were derived. The term “nucleic acid” refers to a deoxyribonucleotide or ribonucleotide in either single- or double-stranded form. The term encompasses nucleic acids, i.e., oligonucleotides, containing known analogs of natural nucleotides that have similar or improved binding properties, for the purposes desired, as the reference nucleic acid. The term also includes nucleic acids which are metabolized in a manner similar to naturally occurring nucleotides or at rates that are improved for the purposes desired. The term also encompasses nucleic-acid-like structures with synthetic backbones. One of skill in the art would recognize how to use a nucleic acid probe for screening of cancer cells in a sample.

Detection of the gene expression level can be conducted in real time in an amplification assay. In one aspect, the amplified products can be directly visualized with fluorescent DNA-binding agents including but not limited to DNA intercalators and DNA groove binders. Because the amount of the intercalators incorporated into the double-stranded DNA molecules is typically proportional to the amount of the amplified DNA products, one can conveniently determine the amount of the amplified products by quantifying the fluorescence of the intercalated dye using conventional optical systems in the art. DNA-binding dye suitable for this application include SYBR green, SYBR blue, DAPI, propidium iodine, Hoeste, SYBR gold, ethidium bromide, acridines, proflavine, acridine orange, acriflavine, fluorcoumanin, ellipticine, daunomycin, chloroquine, distamycin D, chromomycin, homidium, mithramycin, ruthenium polypyridyls, anthramycin, and the like.

In another aspect, other fluorescent labels such as sequence specific probes can be employed in the amplification reaction to facilitate the detection and quantification of the amplified products. Probe-based quantitative amplification relies on the sequence-specific detection of a desired amplified product. It utilizes fluorescent, target-specific probes (e.g., TaqMan® probes) resulting in increased specificity and sensitivity. Methods for performing probe-based quantitative amplification are well established in the art and are taught in U.S. Pat. No. 5,210,015.

For a convenient detection of the probe-target complexes formed during the hybridization assay, the nucleotide probes can be conjugated to a detectable label. Detectable labels suitable for use in methods and compositions described herein include any composition detectable by photochemical, biochemical, spectroscopic, immunochemical, electrical, optical, or chemical means. A wide variety of appropriate detectable labels are known in the art, which include fluorescent or chemiluminescent labels, radioactive isotope labels, enzymatic or other ligands. In preferred embodiments, one will likely desire to employ a fluorescent label or an enzyme tag, such as digoxigenin, B-galactosidase, urease, alkaline phosphatase or peroxidase, avidin/biotin complex.

The detection methods used to detect or quantify the hybridization intensity will typically depend upon the label selected above. For example, radiolabels can be detected using photographic film or a phosphoimager. Fluorescent markers can be detected and quantified using a photodetector to detect emitted light. Enzymatic labels are typically detected by providing the enzyme with a substrate and measuring the reaction product produced by the action of the enzyme on the substrate; and finally colorimetric labels are detected by simply visualizing the colored label.

Any technique for sequencing nucleic acid known to those skilled in the art can be used in the methods of the provided herein. DNA sequencing techniques include classic dideoxy sequencing reactions (Sanger method) using labeled terminators or primers and gel separation in slab or capillary, sequencing by synthesis using reversibly terminated labeled nucleotides, pyrosequencing, 454 sequencing, allele specific hybridization to a library of labeled oligonucleotide probes, sequencing by synthesis using allele specific hybridization to a library of labeled clones is followed by ligation, real time monitoring of the incorporation of labeled nucleotides during a polymerization step, polony sequencing, and SOLiD sequencing. Sequencing of the separated molecules has more recently been demonstrated by sequential or single extension reactions using polymerases or ligases as well as by single or sequential differential hybridizations with libraries of probes. These reactions have been performed on many clonal sequences in parallel including demonstrations in current commercial applications of over 100 million sequences in parallel. These sequencing approaches are used to determine the sequence of nucleic acids in samples.

### B. Protein

In some aspects of the invention protein levels are determined to determine the level of expression of nucleoside transporter in a sample.

Determining the protein level typically involves: a) contacting the protein contained in a sample which contains cancer cells with an agent that specifically bind to a biomarker (examples of the biomarker include but are not limited to the hENTs and hCNTs proteins), and (b) identifying any agent:protein complex so formed. In one aspect of this embodiment, the agent that specifically binds a cancer related protein is an antibody, and the protein is in a biopsy or cell culture, or on cells within the body of a subject.

The agent:protein complex can be a agent:polypeptide or agent:peptide complex.

The formation of the agent:protein complex can be detected directly or indirectly according to standard procedures in the art. In the direct detection method, the agents are supplied with a detectable label and unreacted agents can be removed from the complex; the amount of remaining label thereby indicating the amount of complex formed. For such a method, it is preferable to select labels that remain attached to the agents even during stringent washing conditions. It is preferable that the label does not interfere with the binding reaction. In the alternative, an indirect detection procedure requires the agent to contain a label introduced either chemically or enzymatically. A desirable label generally does not interfere with binding or the stability of the resulting agent:protein complex. However, the label is typically designed to be accessible to an antibody for an effective binding and hence generating a detectable signal.

A wide variety of labels suitable for detecting protein levels are known in the art. Non-limiting examples include radioisotopes, enzymes, colloidal metals, fluorescent compounds, bioluminescent compounds, and chemiluminescent compounds.

The amount of agent:protein complexes formed during the binding reaction can be quantified by standard quantitative assays. As described above, the formation of agent:protein complex can be measured directly by the amount of label remained at the site of binding.

In some embodiments, the cancer related protein is tested for its ability to compete with a labeled analog for binding sites on the specific agent. In this competitive assay, the amount of label captured is inversely proportional to the amount of cancer related protein present in a test sample.

A number of techniques for protein analysis based on the general principles outlined above are available. They include but are not limited to radioimmunoassays, ELISA (enzyme linked immunosorbent assay), “sandwich” immunoassays, immunoradiometric assays, in situ immunoassays (using e.g., colloidal gold, enzyme or radioisotope labels), western blot analysis, immunoprecipitation assays, immunofluorescent assays, and SDS-PAGE.

Automated (computer-aided) image analysis systems known in the art can augment visual examination of tumor samples. A cell or tissue sample can be exposed to detectably-labeled reagents specific for a particular biological marker, and the magnified image of the cell can be then processed, for example, by a computer that receives the image from a charge-coupled device (CCD) or camera such as a television camera. Such a system can be used, for example, to detect and measure expression and activation levels of hENT1 in a sample, or any additional diagnostic biomarkers. Thus, methods described herein can provide more accurate cancer diagnosis and better characterization of gene expression in histologically identified cancer cells, most particularly with regard to expression of tumor marker genes or genes known to be expressed in particular cancer types and subtypes or genes known to alter efficacy of a chemotherapeutic agent. This information can permit a more informed and effective regimen of therapy to be administered, because drugs with clinical efficacy for certain tumor types or subtypes can be administered to patients whose cells are so identified.

Patterns of expression of polypeptides can be detected and quantified using methods known in the art. For example, the pattern of expression of a polypeptide can be detected using biodetection reagents specific for the polypeptide. For example, the biodetection reagent can be an antibody. The term “antibody” as used herein includes all forms of antibodies, including but not limited to recombinant antibodies, chimeric antibodies, single chain antibodies, humanized antibodies, fusion proteins, monoclonal antibodies, polyclonal antibodies, non-human antibodies, fully human antibodies, and antibody fragments. The modifier “monoclonal” indicates the character of the antibody as being obtained from a substantially homogeneous population of antibodies, and is not to be construed as requiring production of the antibody by any particular method. For example, monoclonal antibodies useful for methods described herein be made by the hybridoma method first described by Kohler et al., Nature, 256:495 (1975), or can be made by recombinant DNA methods (see, e.g., U.S. Pat. No. 4,816,567). The “monoclonal antibodies” can also be isolated from phage antibody libraries using the techniques described in Clackson et al., Nature, 352:624-628 (1991) and Marks et al., J. Mol. Biol., 222:581-597 (1991). Chimeric antibodies include those antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity (U.S. Pat. No. 4,816,567; and Morrison et al., Proc. Natl. Acad. Sci. USA, 81:6851-6855 (1984)). “Antibody fragments” include any portion of an intact antibody, preferably comprising the antigen-binding or variable region thereof. Non-limiting examples of antibody fragments include Fab, Fab′, F(ab′)2, and Fv fragments; diabodies; linear antibodies; single-chain antibody molecules; and multispecific antibodies formed from antibody fragment(s). Humanized forms of non-human (e.g., rodent) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit, avian, other mammalian or non-mammalian animals or nonhuman primate having the desired specificity, affinity, and capacity. In some instances, framework region (FR) residues of the human immunoglobulin are replaced by corresponding non-human residues. Furthermore, humanized antibodies can comprise residues that are not found in the recipient antibody or in the donor antibody. These modifications can be made to further refine antibody performance. For further details on humanized antibodies, see Jones et al., Nature 321:522-525 (1986); Riechmann et al., Nature 332:323-329 (1988); and Presta, Curr. Op. Struct. Biol. 2:593-596 (1992).

Methods for generating antibodies against an antigen of interest are known in the art. For example, polyclonal antibodies can be raised in animals by multiple subcutaneous (sc) or intraperitoneal (ip) injections of the relevant antigen and an adjuvant. It can be useful to conjugate the relevant antigen to a protein that is immunogenic in the species to be immunized, e.g., keyhole limpet hemocyanin, serum albumin, bovine thyroglobulin, or soybean trypsin inhibitor using a bifunctional or derivatizing agent, for example, maleimidobenzoyl sulfosuccinimide ester (conjugation through cysteine residues), N-hydroxysuccinimide (through lysine residues), glutaraldehyde, succinic anhydride, SOCl₂, or R₁N═C═NR, where R and R₁ are different alkyl groups. Animals can be immunized against the antigen, immunogenic conjugates, or derivatives by combining, e.g., 100 μg or 5 μg of the protein or conjugate (for rabbits or mice, respectively) with 3 volumes of Freund's complete adjuvant and injecting the solution intradermally at multiple sites. One month later the animals are boosted with one fifth to one tenth of the original amount of peptide or conjugate in Freund's complete adjuvant by subcutaneous injection at multiple sites. Seven to 14 days later the animals are bled and the serum is assayed for antibody titer. Animals are boosted until the titer plateaus. Preferably, the animal is boosted with the conjugate of the same antigen, but conjugated to a different protein and/or through a different cross-linking reagent. Conjugates also can be made in recombinant cell culture as protein fusions. Also, aggregating agents such as alum are suitably used to enhance the immune response.

Methods for producing monoclonal antibodies are known in the art, and include, for example, the hybridoma method. In the hybridoma method, a mouse or other appropriate host animal, such as a hamster or rabbit, is immunized as hereinabove described to elicit lymphocytes that produce or are capable of producing antibodies that will specifically bind to the protein used for immunization. Alternatively, lymphocytes can be immunized in vitro. Lymphocytes then are fused with myeloma cells using a suitable fusing agent, such as polyethylene glycol, to form a hybridoma cell (Goding, Monoclonal Antibodies: Principles and Practice, pp. 59-103 (Academic Press, 1986)). The hybridoma cells thus prepared are seeded and grown in a suitable culture medium that preferably contains one or more substances that inhibit the growth or survival of the unfused, parental myeloma cells. For example, if the parental myeloma cells lack the enzyme hypoxanthine guanine phosphoribosyl transferase (HGPRT or HPRT or other similar marker gene), the culture medium for the hybridomas typically will include hypoxanthine, aminopterin, and thymidine (HAT medium), which substances prevent the growth of HGPRT-deficient cells. Culture medium in which hybridoma cells are growing is assayed for production of monoclonal antibodies directed against the antigen. Preferably, the binding specificity of monoclonal antibodies produced by hybridoma cells is determined by immunoprecipitation or by an in vitro binding assay, such as radioimmunoassay (RIA) or enzyme-linked immunoabsorbent assay (ELISA). After hybridoma cells are identified that produce antibodies of the desired specificity, affinity, and/or activity, the clones can be subcloned by limiting dilution procedures and grown by standard methods (Goding, Monoclonal Antibodies: Principles and Practice, pp. 59-103 (Academic Press, 1986)). The monoclonal antibodies secreted by the subclones are suitably separated from the culture medium, ascites fluid, or serum by conventional antibody purification procedures such as, for example, protein A-Sepharose, hydroxylapatite chromatography, gel electrophoresis, dialysis, or affinity chromatography. DNA encoding the monoclonal antibodies is readily isolated and sequenced using conventional procedures (e.g., by using oligonucleotide probes that are capable of binding specifically to genes encoding the heavy and light chains of murine antibodies along with techniques such as PCR). The hybridoma cells serve as a preferred source of such DNA. Once isolated, the DNA can be placed into expression vectors, which are then transfected into host cells such as E. coli cells, simian COS cells, Chinese Hamster Ovary (CHO) cells, or myeloma cells that do not otherwise produce antibody protein, to obtain the synthesis of monoclonal antibodies in the recombinant host cells. Methods for further manipulating antibodies based on sequence modification, chimerization, humanization, structural modeling, and fragmentation are known in the art.

As an alternative to humanization, human antibodies can be generated. For example, it is now possible to produce transgenic animals (e.g., mice) that are capable, upon immunization, of producing a full repertoire of human antibodies in the absence of endogenous immunoglobulin production. For example, it has been described that the homozygous deletion of the antibody heavy-chain joining region (J H) gene in chimeric and germ-line mutant mice results in complete inhibition of endogenous antibody production. Transfer of the human germ-line immunoglobulin gene array in such germ-line mutant mice will result in the production of human antibodies upon antigen challenge. See, e.g., Jakobovits et al., Proc. Natl. Acad. Sci. USA, 90:2551 (1993); Jakobovits et al., Nature, 362:255-258 (1993); Bruggermann et al., Year in Immuno., 7:33 (1993); and U.S. Pat. Nos. 5,591,669, 5,589,369 and 5,545,807. Alternatively, phage display technology (McCafferty et al., Nature 348:552-553 (1990)) can be used to produce human antibodies and antibody fragments in vitro, from immunoglobulin variable (V) domain gene repertoires from unimmunized donors. According to this technique, antibody V domain genes are cloned in-frame into either a major or minor coat protein gene of a filamentous bacteriophage, such as M113 or fd, and displayed as functional antibody fragments on the surface of the phage particle. Because the filamentous particle contains a single-stranded DNA copy of the phage genome, selections based on the functional properties of the antibody also result in selection of the gene encoding the antibody exhibiting those properties. Thus, the phage mimics some of the properties of the B-cell. Phage display can be performed in a variety of formats; for their review see, e.g., Johnson, Kevin S. and Chiswell, David J., Current Opinion in Structural Biology 3:564-571 (1993). Several sources of V-gene segments can be used for phage display. Clackson et al., Nature, 352:624-628 (1991) isolated a diverse array of anti-oxazolone antibodies from a small random combinatorial library of V genes derived from the spleens of immunized mice. A repertoire of V genes from unimmunized human donors can be constructed and antibodies to a diverse array of antigens (including self-antigens) can be isolated essentially following the techniques described by Marks et al., J. Mol. Biol. 222:581-597-(1991), or Griffith et al., EMBO J. 12:725-734 (1993). See, also, U.S. Pat. Nos. 5,565,332 and 5,573,905. Human antibodies can also be generated by in vitro activated B cells (see U.S. Pat. Nos. 5,567,610 and 5,229,275).

Various techniques have been developed for the production of antibody fragments comprising one or more antigen binding regions. Traditionally, these fragments were derived via proteolytic digestion of intact antibodies (see, e.g., Morimoto et al., Journal of Biochemical and Biophysical Methods 24:107-117 (1992); and Brennan et al., Science, 229:81 (1985)). However, these fragments can now be produced directly by recombinant host cells. For example, the antibody fragments can be isolated from the antibody phage libraries discussed above. Alternatively, Fab′-SH fragments can be directly recovered from E. coli and chemically coupled to form F(ab′)2 fragments (Carter et al., Bio/Technology 10:163-167 (1992)). According to another approach, F(ab′)2 fragments can be isolated directly from recombinant host cell culture. Other techniques for the production of antibody fragments will be apparent to the skilled practitioner. In other embodiments, the antibody of choice is a single chain Fv fragment (scFv). See WO 93/16185; U.S. Pat. No. 5,571,894; and U.S. Pat. No. 5,587,458. The antibody fragment can also be a “linear antibody”, e.g., as described in U.S. Pat. No. 5,641,870 for example. Such linear antibody fragments can be monospecific or bispecific.

Alternatively, the biodetection reagents can be aptamers. Aptamers are nucleic acids or peptides that bind non-nucleic acid target molecules. Accordingly, binding agents suitable for methods described herein also include aptamers. The aptamers bind to targets for determining nucleoside transporter levels. Aptamers can be selected by any means now known or later discovered. For example, the aptamer can be selected for its ability to bind to hENT 1.

The aptamer can be selected by a SELEX, or Systematic Evolution of Ligands by EXponential enrichment, process. As used herein, SELEX is a process for selecting aptamers that have high binding affinities for a particular target. In brief, the process generally involves (a) generation of a large library of aptamer oligonucleotides (e.g., having as much as 10¹⁵ different sequences); (b) exposing the library to the target of interest; (c) eliminating the library members that do not bind to the target; (d) amplifying the library members that bind to the target, creating a library enriched for aptamers that bind to the target; (e) repeating steps (b) through (d) as many as 15 times or more to isolate the aptamers that bind the most tightly to the target. As used herein, the SELEX process also includes various modifications now known (see, e.g., U.S. Pat. Nos. 5,472,841; 5,503,978; 5,567,588; 5,582,981; 5,637,459; 5,683,867; 5,705,337; 5,712,375; and 6,083,696) or later discovered. Similar processes utilizing rounds of selection or screening can be similarly applied in the evaluation of other potential biodetection reagents, including but not limited to peptides, peptide mimetics, proteins, small molecules, and antibodies.

In some embodiments, polypeptides are quantified by image analysis using a suitable primary antibody against biomarkers, including but not limited to hENTs, or hCNTs., detected directly or using an appropriate secondary antibody (such as rabbit anti-mouse IgG when using mouse primary antibodies) and/or a tertiary avidin (or Strepavidin) biotin complex (“ABC”).

In some embodiments, the antibody against hENT1 is an antibody that includes a variable heavy chain complementarity determining region 1 (VH CDR1) that includes an amino acid sequence at least 90%, 92%, 95%, 97% 98%, 99% or more identical to the amino acid sequence GYTFTDYE (SEQ ID NO: 1); a variable heavy chain complementarity determining region 2 (VH CDR2) that includes an amino acid sequence at least 90%, 92%, 95%, 97% 98%, 99% or more identical to the amino acid sequence IDPETGAI (SEQ ID NO: 2) or the amino acid sequence IDPETGKT (SEQ ID NO: 3); and a variable heavy chain complementarity determining region 3 (VH CDR3) that includes an amino acid sequence at least 90%, 92%, 95%, 97% 98%, 99% or more identical to the amino acid sequence TREFTY (SEQ ID NO: 4) or the amino acid sequence TRELTY (SEQ ID NO: 5).

In some embodiments, the antibody against hENT1 is an antibody that includes a variable heavy chain complementarity determining region 1 (VH CDR1) sequence comprising the amino acid sequence GYTFTDYE (SEQ ID NO: 1), a variable heavy chain complementarity determining region 2 (VH CDR2) sequence comprising the amino acid sequence IDPETGAI (SEQ ID NO: 2) or the amino acid sequence IDPETGKT (SEQ ID NO: 3), and a variable heavy chain complementarity determining region 3 (VH CDR3) sequence comprising the amino acid sequence TREFTY (SEQ ID NO: 4) or the amino acid sequence TRELTY (SEQ ID NO: 5).

In some embodiments, the antibody against hENT1 is an antibody that includes a variable light chain complementarity determining region 1 (VL CDR1) sequence that includes an amino acid sequence at least 90%, 92%, 95%, 97% 98%, 99% or more identical to the amino acid sequence QSLLFSNGKTY (SEQ ID NO: 6), a variable light chain complementarity determining region 2 (VL CDR2) sequence that includes an amino acid sequence at least 90%, 92%, 95%, 97% 98%, 99% or more identical to the amino acid sequence LVS (SEQ ID NO: 7), and a variable light chain complementarity determining region 3 (VL CDR3) sequence that includes an amino acid sequence at least 90%, 92%, 95%, 97% 98%, 99% or more identical to the amino acid sequence VQGTHFPWT (SEQ ID NO: 8).

In some embodiments, the antibody against hENT1 is an antibody that includes a variable light chain complementarity determining region 1 (VL CDR1) sequence that includes the amino acid sequence QSLLFSNGKTY (SEQ ID NO: 24), a variable light chain complementarity determining region 2 (VL CDR2) sequence that includes the amino acid sequence LVS (SEQ ID NO: 25), and a variable light chain complementarity determining region 3 (VL CDR3) sequence that includes the amino acid sequence VQGTHFPWT (SEQ ID NO: 26).

In some embodiments, the antibody against hENT1 is an antibody that includes a variable heavy chain that includes an amino acid sequence that is at least 90%, 92%, 95%, 97% 98%, 99% or more identical to the amino acid sequence shown below in SEQ ID NO: 9 or SEQ ID NO: 10. In some embodiments, the antibody against hENT1 is an antibody that includes a variable light chain that includes an amino acid sequence that is at least 90%, 92%, 95%, 97% 98%, 99% or more identical to the amino acid sequence shown below in SEQ ID NO: 11 or SEQ ID NO: 12.

```
  (SEQ ID NO: 9)
VH amino acid sequence 1

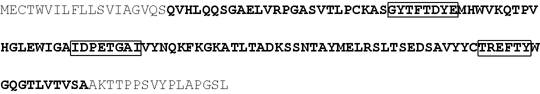

(SEQ ID NO: 10)
VH amino acid sequence 2

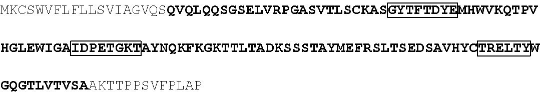

  (SEQ ID NO: 11)
VL amino acid sequence 1

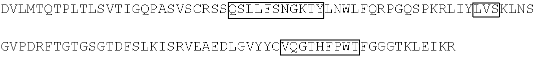

  (SEQ ID NO: 12)
VL amino acid sequence 2

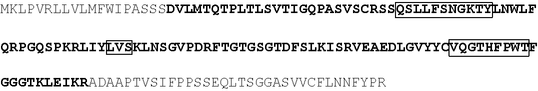

```

In some embodiments, the antibody against hENT1 is an antibody that includes a variable heavy chain that includes the amino acid sequence shown below in SEQ ID NO: 9 or SEQ ID NO: 10. In some embodiments, the antibody against hENT1 is an antibody that includes a variable light chain that includes the amino acid sequence shown below in SEQ ID NO: 11 or SEQ ID NO: 12.

The amount of target protein can be quantified by measuring the average optical density of the stained antigens. Concomitantly, the proportion or percentage of total tissue area stained can be readily calculated, for example as the area stained above a control level (such as an antibody threshold level) in the second image. Following visualization of nuclei containing biomarkers, the percentage or amount of such cells in tissue derived from patients after treatment are compared to the percentage or amount of such cells in untreated tissue. As used herein, “determining” a pattern of expression of polypeptides is understood broadly to mean merely obtaining the information on such polypeptide(s), either through direct examination or indirectly from, for example, a contract diagnostic service.

Cancer tissue sections taken from patients can be analyzed by immuno-staining for expression of proteins such as hENTs or hCNTs. In some embodiments, cancer, tumor or any tissue suspected of being cancerous or tumorigenic is analyzed by immunohistochemistry for expression of hENT1. In some embodiments, cancer, tumor or any tissue suspected of being cancerous or tumorigenic is analyzed by immunohistochemistry for expression of hENT1. These measurements can be accomplished, for example, by using tissue microarrays. Tissue microarrays are a well-validated method to rapidly screen multiple tissue samples under uniform staining and scoring conditions. (Hoos et al., 2001, Am J Pathol. 158: 1245-51). Scoring of the stained arrays can be accomplished by an automated system that accurately quantified the staining observed. The results of this analysis identify biomarker levels and can be used to predict patient outcome following treatment, such as gemcitabine or gemcitabine-5′-elaidate therapies.

### VII. Methods for Classifying and Treating Cancer

In another aspect the present invention provides methods useful for classifying samples based upon the levels of expression of the nucleoside transporters. In some embodiments, the classification is useful for determining which individuals will respond to particular compounds of the invention. In some embodiments, methods described herein are useful for identifying cancers that respond to gemcitabine. In some embodiments, methods of this invention can be used to identify cancers that do not respond to gemcitabine. In further embodiments methods described herein are useful for identifying a subject with cancer to not receive treatment with gemcitabine. In some embodiments, methods described herein are used for identifying a subject with cancer to receive treatment with a gemcitabine analog. In some embodiments, methods described herein are useful for identifying a subject with cancer to receive treatment with gemcitabine-5′-elaidate. In some embodiments, methods described herein further include determining the dosing regimen for the cancer subject.

In some embodiments, the nucleoside transporter level of a sample is determined and classified. The classification of the sample is then matched to an appropriate therapy. This therapy includes a particular drug regime. The drug regime is communicated to a patient. The patient follows the instructions, takes the appropriate drug and the patient's cancer is successfully treated.

### A. Transporter Expression Levels

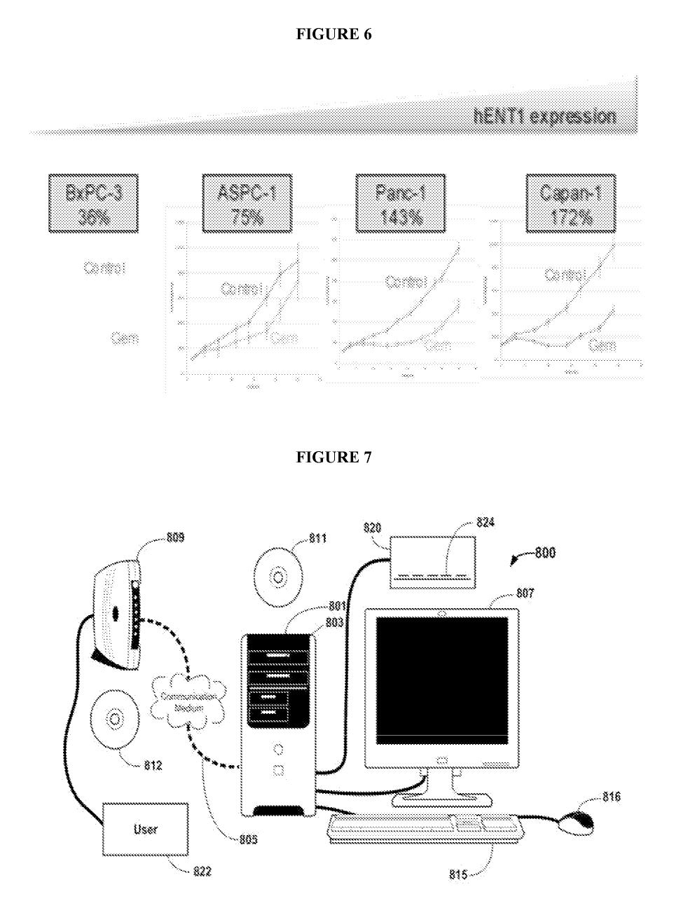

There are many nucleoside transporters and each may move nucleoside analog drugs across the membrane with unique kinetics. A cancer cell which has lower levels nucleoside transporters will be less efficient at transporting these drugs into the cells, reducing the efficacy of the drug. For example a tumor that has low levels of hENT1 is less sensitive to treatment with gemcitabine. FIG. 6 depicts the correlation of hENT1 mRNA levels and sensitivity to gemcitabine in xenograft mouse models.

In some embodiments, the invention provides for methods of classifying or ranking samples based on expression levels of nucleoside transporters. In some embodiments, the nucleoside transporter level of a sample is ranked as “high,” “normal,” or “low.” In some embodiments, the classification or ranking is relative to a statistical distribution of control expression levels. In some embodiments, the classification or ranking is relative to a control sample obtained from the subject. In some embodiment the expression levels of hENT1 is classified or ranked relative to a statistical distribution of control expression levels. In some embodiment the expression levels of hENT1 is classified or ranked relative to an expression levels from a control sample obtained from the subject.

Because of overlapping substrate specificities among different members of ENTs, prospective patients in some embodiments may be tested for low levels of hENT family proteins other than hENT1. In some embodiments, a patent may be tested for one or more of hENT family proteins. Examples of hENT family proteins and their analogs include, but are not limited to, hENT2, hENT2, hENT3, and hENT4.

A patient may be tested for low expression level of one or more hCNTs. In some embodiments, CNT test is performed either independently of or in addition to measuring ENT level. Examples of hCNTs and its analog include, but are not limited to hCNT1, hCNT2, and hCNT3.

Methods described herein are useful for a subject with low uptake rate for hydrophilic nucleoside analogs, such as gemcitabine. Methods disclosed herein are not limited to known nucleoside transporters such as hENTs or hCNTs but are applicable to a prospective patient with low uptake rate due to lower than what is considered as clinically normal expression level for any particular nucleoside transporter.

In some embodiments, other biological characteristics known to affect drug uptake or metabolism are considered in selecting subjects for the effective administration of compositions described herein. Non-limiting examples of other biological characteristics includes single nucleotide polymorphism, complete blood count with differential, expression pattern and enzymatic activities of cytochrome p450 isoforms, gender, ethnicity, age, body weight, family medical history or prior treatment history.

In some embodiments, the patient is screened for nucleoside transporter polymorphisms or genetic mutations that correlate to lower levels of transport of gemcitabine across the membrane.

In some embodiments, expression of hENT1 in tumor tissue is measured in a clinical trial. Patients are categorized into hENT1-high or hENT1-low groups according to methods, control samples, and comparison samples described herein. In a clinical setting, trial is conducted to confirm (1) that low pancreatic tumor hENT1 expression is associated with poor outcome after gemcitabine therapy, and (2) that gemcitabine-5′-elaidate or has superior efficacy in hENT1-low patients compared with gemcitabine.

In some embodiments, designations such as “high” or “low” are used to indicate expression level of a gene. In some embodiment the expression of the gene is measured by mRNA levels. In some embodiments, determination of high or low expression level in a patient sample, such as a tumor biopsy, is made in reference to a normal tissue sample obtained from the patient. In some embodiments, a reference level clinically accepted as normal level in a standardized test can be used to determine the level of expression in tumor sample. In some embodiments, determination of high or low expression level is made in reference to a control sample obtained from a control subject other than the patient. A control subject can be a person who is deemed to be a healthy person sharing similar ethnic, age, and gender identity with the patient. In some embodiments, “low” expression is mRNA levels less than about 1.1, 1.2, 1.3, 1.5, 1.7, 2, 2.2, 2.5, 2.7, 3, 5, 7, 10, 20, 50, 70, 100, 200, 500, 1000 times or less than 1000 times to that of what is considered as clinically normal or to the expression level obtained from control tissue.

In some embodiments, designation such as “high” or “low” are used to indicate expression level of a nucleoside transporter protein. In some embodiment the expression of the protein is measured by immunohistochemistry. In some embodiments, determination of high or low expression level in a patient sample, such as a tumor biopsy, is made in reference to a normal tissue sample obtained from the patient. In some embodiments, a reference level clinically accepted as normal level in a standardized test can be used to determine the level of expression in tumor sample. In some embodiments, determination of high or low expression level is made in reference to a control sample obtained from a control subject other than the patient. A control subject can be a person who is deemed to be a healthy person sharing similar ethnic, age, and gender identity with the patient. In some embodiments, “low” expression is mRNA levels less than about 1.1, 1.2, 1.3, 1.5, 1.7, 2, 2.2, 2.5, 2.7, 3, 5, 7, 10, 20, 50, 70, 100, 200, 500, 1000 times or less than 1000 times to that of what is considered as clinically normal or to the expression level obtained from control tissue.

In some embodiments, the criteria of determine low or high are based on the number of positive staining cells and/or the intensity of the staining, wherein the staining is an indicator of nucleosides transporter protein. In some embodiments, the score is low if less than 50% cells have positive staining In some embodiments, the score is low if less than 1%, 5%, 10%, 15%, 20%, 25%, 30%, 35%, 40%, 45%, or 50%, cells have positive staining. In some embodiments, the score is low if the staining is 1%, 5%, 10%, 15%, 20%, 25%, 30%, 35%, 40%, 45%, or 50% less intense than positive control staining.

In some embodiments, the criteria of determine low or high are based on the number of positive staining cells and/or the intensity of the staining, wherein the staining is an indicator of nucleosides transporter protein. In some embodiments, the score is high if more than 50% cells have positive staining. In some embodiments, the score is high if more than 1%, 5%, 10%, 15%, 20%, 25%, 30%, 35%, 40%, 45%, or 50%, cells have positive staining. In some embodiments, the score is high if more than 40%, 45%, 50%, 55%, 60%, 65%, 70%, 75%, 80%, 85%, or 90%, cells have positive staining. In some embodiments, the score is high if the staining is as intense as positive control staining. In some embodiments, the score is high if the staining is 80%, 85%, or 90% as intense as positive control staining.

In some embodiments, the scoring is based on an “H-score.” An H-score is obtained by the formula: 3×percentage of strongly staining cells+2×percentage of moderately staining cells+percentage of weakly staining cells, giving a range of 0 to 300.

In some aspects strongly staining, moderately staining, and weakly staining are descriptions well known to those in the art. In some aspects strongly staining, moderately staining, and weakly staining are calibrated levels of staining, wherein a range is established and the intensity of staining is binned within the range. In some embodiments, strong staining is staining above the 75th percentile of the intensity range, moderate staining is staining from the 25th to the 75th percentile of the intensity range, and low staining is staining is staining below the 25th percentile of the intensity range. In some aspects one skilled in the art, and familiar with a particular staining technique, adjusts the bin size and defines the staining categories.

In some embodiments, the label high hENT1 staining is assigned where greater than 50% of the cells stained exhibited strong reactivity, the label no hENT1 staining is assigned where no staining was observed in less than 50% of the cells stained, and the label low hENT1 staining is assigned for all of other cases.

In some embodiments, the assessment and scoring of the hENT1 expression level in a sample, patient, etc., is performed by one or more experienced clinicians, i.e., those who are experienced with hENT1 expression and hENT1 staining patterns. For example, in some embodiments, the clinician(s) is blinded to clinical characteristics and outcome for the samples, patients, etc. being assessed and scored.

The applications and uses of the methods for detecting levels of nucleoside transporters described herein can produce one or more results useful to diagnose or classify a disease state of an individual or individual's cancer or tumor. In some embodiments, a method of classifying a tumor comprises reviewing or analyzing data relating to the presence and/or the concentration level of a nucleoside transporter in a sample.

### B. Matching Expression Levels to an Appropriate Therapy

Provided herein are methods for predicting an individual's response to cancer therapy, including a response by human cancer patients. Methods for matching a particular chemotherapeutic agent to particular individual based on predicted efficacy are further provided.

In some embodiments, hydrophobic gemcitabine analogs are administered to subjects shown to have decreased levels of hENT1, such as a subject with decreased levels of compared to normal individuals. In some embodiments, gemcitabine analogs are administered to subjects shown to have low levels of hENT1. In some embodiments, a subject having decreased levels of hENT1 has his or her cancer ameliorated with increased efficacy by treatment with a gemcitabine analog such as gemcitabine-5′-elaidic acid ester.

In some embodiments, hydrophobic gemcitabine analogs are administered to subjects who have provided samples which are classified as having “low” levels of hENT1. In some embodiments, the individual has his or her cancer ameliorated with increased efficacy by treatment with a gemcitabine analog such as gemcitabine-5′-elaidic acid ester.

In some embodiments, improved amelioration of a cancer in a subject is achieved by using a method wherein levels of hENT1 in a subject are measured and a therapeutically effective amount of gemcitabine analog is administered to the subject. In some embodiments, levels of hENT1 in a subject are utilized as indications for the predicted efficacy of gemcitabine analog in the patient. In some embodiments, a determination is made whether the subject has low levels of hENT1 prior to administration of the gemcitabine analog.

In some embodiments, an insurance company or health care provider determines an acceptable protocol for treatment based upon nucleoside transporter levels.

In some embodiments, hCNTs or hENTs other than hENT1 are measured in various cancer described herein. Any low expression level, if detected, is then correlated with a type of cancer in which the low level expression is identified. In some embodiments, the correlated data set is used to identify patients or tumor groups that potentially benefit from methods and compositions described herein. In some embodiments, the correlated date is cross-indexed with known data on gemcitabine response to identify patients or tumor groups poorly responding to gemcitabine treatment and has low expression level of one or more hCNTs or hENTs.

In some aspects low expression of hENT1 on pancreatic tumor cells linked to poor survival after gemcitabine therapy. In addition, hENT1 deficient tumor cells are resistant to pyrimidine nucleoside analogs in vitro. For example, hENT1 mRNA levels are co-related to the sensitivity to gemcitabine in xenograft mouse models. The higher expression level of hENT1 mRNA is associated with better response to gemcitabine.

Low levels of tumor hENT1 expression have been shown to correlate with poor survival outcomes after gemcitabine therapy in pancreatic and lung cancer patients. In patients with pancreatic cancer, low levels of hENT1 expression have been shown to correlate with poor outcome after gemcitabine therapy. Up to two-thirds of pancreatic cancer patients may have limited cellular uptake of gemcitabine, due to deficient expression of hENT1. Approximately 50% of pancreatic cancer patients have been shown to have low tumor expression of hENT1. hENT1 levels predict also outcome in lung cancer patients treated with gemcitabine-containing chemotherapy.

### C. Transmitting Information Regarding Nucleoside Transporter Levels

In some embodiments, an individual or individuals are informed about their nucleoside transporter levels directly by a company that tests samples. In some embodiments, an individual or individuals are informed about their nucleoside transporter levels by their physicians. In some embodiments, an individual or individuals are informed about their nucleoside transporter levels via a secure website. In some embodiments, an individual or individuals are only informed about a classification of their sample or are only informed about the recommended drug and dosage. In some embodiments, an individual or individuals cannot be informed because of incapacitation. In such instances the family or legal guardian is informed.

In some embodiments, patients are informed about their nucleoside transporter levels in combination with information about the proper drug and dosage. In some embodiments, a chemotherapeutic drug is labeled to provide instructions regarding proper dosage given a patient's nucleoside transporter level.

A conclusion based on a review or analysis of the data can be provided to a patient, a health care provider or a health care manager. In some embodiments, the conclusion is based on the review or analysis of data regarding a disease diagnosis. In some embodiments, the conclusion is based on the review or analysis of data regarding which drug to treat a subject with. It is envisioned that in some embodiments providing a conclusion to a patient, a health care provider or a health care manager includes transmission of the data over a network. In some embodiments, raw data or partially interpreted data is shared via transmission of the data over a network. Accordingly systems and methods using the scanning sensing systems and methods described herein are provided.

One aspect of the invention is a method comprising screening patient samples for the presence or absence of a biologically nucleoside transporters to produce data regarding the sample, collecting the sample data, providing the data to a patient, a health care provider or a health care manager for making a conclusion based on review or analysis of the data regarding a tumor classification. In some embodiments, the conclusion is provided to a patient, a health care provider or a health care manager includes transmission of the data over a network.

FIG. 7 is a block diagram showing a representative example logic device through which reviewing or analyzing data relating to the present invention can be achieved. Such data can be in relation to a disease, disorder or condition in an individual. FIG. 7 shows a computer system (or digital device) 800 connected to an apparatus 820 for use with the scanning sensing system 824 to, for example, produce a result. The computer system 800 may be understood as a logical apparatus that can read instructions from media 811 and/or network port 805, which can optionally be connected to server 809 having fixed media 812. The system shown in FIG. 7 includes CPU 801, disk drives 803, optional input devices such as keyboard 815 and/or mouse 816 and optional monitor 807. Data communication can be achieved through the indicated communication medium to a server 809 at a local or a remote location. The communication medium can include any means of transmitting and/or receiving data. For example, the communication medium can be a network connection, a wireless connection or an internet connection. Such a connection can provide for communication over the World Wide Web. It is envisioned that data relating to the present invention can be transmitted over such networks or connections for reception and/or review by a party 822. The receiving party 822 can be but is not limited to a patient, a health care provider or a health care manager.

In some embodiments, a computer-readable medium includes a medium suitable for transmission of a result of an analysis of an environmental or biological sample. The medium can include a result regarding a disease condition or state of a subject, wherein such a result is derived using the methods described herein.

### VIII. Compounds of the Invention

Compounds for the treatment of cancer based upon levels of nucleoside transporters are disclosed. One skilled in the art will recognize that some embodiments of this invention are applicable to any compound which relies on nucleoside transporters to transit the plasma membrane. As such some embodiments of the present invention include recommendations to a patient regarding drug efficacy wherein the drug is known to transit the plasma membrane through a nucleoside transporter and the level of said nucleoside transporter has been determined.

Some chemotherapeutic agents have been modified reduce their reliance on cellular transporters to transit a membrane. For individuals with a reduced capacity for a chemotherapeutic agent to transit a relevant cell membrane via transporters these modified chemotherapeutic agents are more efficacious than agents that require transporters.

Methods for using additional agents to manipulate the transporters to increase the efficacy of chemotherapeutic agents are further disclosed.

### A. Hydrophilic Nucleosides Drugs

Hydrophilic nucleosides transit the plasma membrane through specific channels, or nucleoside transporters. These drugs are most useful for treatment of cancers that express nucleoside transporters, specifically those transporters that allow the particular drug to transit the membrane. Some examples of hydrophilic nucleoside drugs are Capecitabine, Cladribine, Clofarabine, Cytarabine, Fludarabine, and Gemcitabine.

In some embodiments, an individual is advised to use Capecitabine based upon the level (e.g., “high”) of nucleoside transporters in a sample from the patient. In some embodiments, an individual is advised to not to use Capecitabine based upon the level (e.g., “low”) of nucleoside transporters in a sample from the patient.

Capecitabine (5′-deoxy-S—N-[(pentoxy)carbonyl]-cytidine) is a pyrimidine nucleoside. Capecitabine is a prodrug that is metabolized by carboxylesterase to 5′-deoxy-5-fluorocytidine after oral administration. 5′-Deoxy-5-fluorocytidine is deaminated by cytidine deaminase to 5′-deoxy-5-fluorouridine. hENT1 mediates the uptake of 5′-deoxy-5-fluorouridine. A metabolite of capecitabine, 5′-deoxy-5-fluorouridine monophosphate, inhibits thymidylate synthase and the 5′-deoxy-5-fluorouridine triphosphate is incorporated into DNA. The last activation step is catalyzed by thymidine phosphorylase, which converts 5′-deoxy-5-fluorouridine into 5-fluorouracil. Thymidine phosphorylase is highly expressed in tumor tissues and is associated with resistance to conventional 5-fluorouracil treatment in several gastrointestinal tumors, in particular colon cancer. Capecitabine has shown activity in metastatic colorectal cancer that is comparable to that of 5-fluorouracil combined with leucovorin. Capecitabine has activity against metastatic breast cancer that has progressed after docetaxel or anthracyclines.

In some embodiments, an individual is advised to use Cladribine based upon the level (e.g., “high”) of nucleoside transporters in a sample from the patient. In some embodiments, an individual is advised to not to use Cladribine based upon the level (e.g., “low”) of nucleoside transporters in a sample from the patient.

Cladribine (2-CdA, 2-chloro-2′-deoxyadenosine) has a chlorine substitution at the 2 position of the adenine moiety. Cladribine is resistant to deamination by adenosine deaminase. Cladribine enters cells via hENT1, hENT2 and hCNT3 and is converted into the active form 2-CdATP by the combined action of dCK and cellular nucleotide kinases. Cladribine inhibits DNA replication and repair as well as ribonucleotide reductase thereby reducing deoxyribonucleotide synthesis. Exposure to cladribine is cytotoxic to dividing cells because of the inhibition of replicative DNA synthesis and to resting cells because of the inhibition of DNA repair processes and alteration of mitochondrial function or integrity. Cladribine has been shown to be active in low-grade lymphomas chronic lymphocytic leukemia and hairy cell leukemia.

In some embodiments, an individual is advised to use Clofarabine based upon the level (e.g., “high”) of nucleoside transporters in a sample from the patient. In some embodiments, an individual is advised to not to use Clofarabine based upon the level (e.g., “low”) of nucleoside transporters in a sample from the patient.

Clofarabine (Cl-FaraA, 2-chloro-9-(2′-deoxy-2′-fluoro-β-d-arabinofuranosyl)adenine) has activity against both epithelial and hematologic malignancies. Substitution at position 2 of the base with a halogen confers resistance to deamination. Substitution of fluorine on the arabinosyl sugar prevents degradation by bacterial phosphorylase and allows oral administration. Clofarabine enters cells via hENT1, hENT2, hCNT2 and possibly also hCNT3. Clofarabine is metabolized to its mono-, di- and triphosphates by dCK and nucleotide kinases. Cl-FaraATP is incorporated into replicating DNA, which terminates chain elongation, and inhibits ribonucleotide reductase, which decreases intracellular deoxynucleotide pools.

In some embodiments, an individual is advised to use Gemcitabine based upon the level (e.g., “high”) of nucleoside transporters in a sample from the patient. In some embodiments, an individual is advised to not to use Gemcitabine based upon the level (e.g., “low”) of nucleoside transporters in a sample from the patient.

Gemcitabine (2′2′-difluorodeoxycytidine or dFdC) is a deoxycytidine analog. Gemcitabine is an analog of cytarabine, which was modified at the 2′-position of the ribose ring by substitution of two fluorine atoms to give gemcitabine. Gemcitabine is a hydrophilic nucleoside drugs.

Gemcitabine can kill cells. Gemcitabine exhibits cell phase specificity, primarily killing cells undergoing DNA synthesis (S-phase) and also blocking the progression of cells through the G1/S-phase boundary. The cytotoxic effect of gemcitabine is attributed to a combination of two actions of the diphosphate and the triphosphate nucleosides, which leads to inhibition of DNA synthesis. First, gemcitabine diphosphate inhibits ribonucleotide reductase, which is responsible for catalyzing the reactions that generate the deoxynucleoside triphosphates for DNA synthesis. Inhibition of this enzyme by the diphosphate nucleoside causes a reduction in the concentrations of deoxynucleotides, including dCTP. Second, gemcitabine triphosphate competes with dCTP for incorporation into DNA. The reduction in the intracellular concentration of dCTP (by the action of the diphosphate) enhances the incorporation of gemcitabine triphosphate into DNA (self-potentiation). After the gemcitabine nucleotide is incorporated into DNA, only one additional nucleotide is added to the growing DNA strands. After this addition, there is inhibition of further DNA synthesis. DNA polymerase epsilon is unable to remove the gemcitabine nucleotide and repair the growing DNA strands (masked chain termination).

Gemcitabine is a hydrophilic molecule. The entry of gemcitabine into cells, e.g., tumor cells, is dependent upon the expression of membrane transporter proteins, particularly Human equilibrative nucleoside transporter 1 (hENT1). Mackey et al Cancer Res (1998); Damaraju et al Nucl Acids (2009). In some embodiments, determining the level of hENT1 in a sample from a patient is instructive for the treatment of a patient using gemcitabine.

Gemcitabine is the current standard treatment for advanced pancreatic cancer, and is also used in combination with other chemotherapy agents for the treatment of other cancers, including ovarian, non-small cell lung, head and neck, and breast cancers. Gemcitabine has activity against metastatic bladder cancer, and Gemcitabine combined with cisplatin has been used as treatment for metastatic bladder cancer.

In some aspects the present invention provides for administering gemcitabine to an individual who does not have low hENT1 levels. In some aspects the present invention provides for recommending the administration of gemcitabine to an individual who does not have low hENT1 levels. In some aspects the present invention provides for instruction regarding doses of gemcitabine for an individual based upon the level of hENT1 in a sample derived from the individual.

### B. Lipophilic Nucleoside Analogs

In another aspect lipophilic nucleoside analogs are of interest because these may enter cells independent of the nucleoside transporters by facilitated diffusion across the lipid membrane. A family of lipophilic gemcitabine analogs having an elaidic fatty acid esterified at the 5′ position has been produced. Some embodiments of the gemcitabine derivatives are depicted by Formula (I):

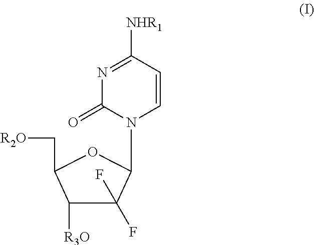

wherein R₁, R₂ and R₃ are independently selected from hydrogen and C18 and C20 saturated and monounsaturated acyl groups, with the proviso that R₁, R₂ and R₃ cannot all be hydrogen, or a pharmaceutically acceptable salt thereof as the active ingredient. Additional gemcitabine derivatives are disclosed in U.S. Pat. No. 6,384,019.

In some embodiments, the gemcitabine derivative of Formula (I) has R₁ and R₃ as hydrogen and R₂ is a C18 or C20 saturated or monounsaturated acyl group.

Gemcitabine has three derivatisable functions, namely the 5′ and 3′ hydroxyl groups and the N4 amino group. Each group can selectively be transformed into an ester or amide derivative, but di-adducts (di-esters or ester-amides) and tri-adducts may be formed as well. In the case of the di- and tri-adducts the acyl substituent groups need not necessarily be the same.

In some embodiments, the mono-acyl derivatives, i.e. with two of R₁, R₂ and R₃ being hydrogen, are preferred for use as the active ingredient of the present pharmaceutical composition. It is especially preferred that the monosubstitution with the acyl group should be in the 3′-O and 5′-O positions of the sugar moiety, with 5′-O substitution being most preferred.

The double bond of the mono-unsaturated acyl groups may be in either the cis or the trans configuration, although the therapeutic effect may differ depending on which configuration is used.

The position of the double bond in the mono-unsaturated acyl groups also seems to affect the activity. In some embodiments, it is preferred to use esters or amides having their unsaturation in the ω-9 position. In the ω system of nomenclature, the position ω of the double bond of a monounsaturated fatty acid is counted from the terminal methyl group, so that, for example, eicosenoic acid (C20:1 ω9) has 20 carbon atoms in the chain and a single double bond is formed between carbon 9 and 10 counting from the methyl end of the chain. In some embodiments, it is preferred to use esters, ester-amides and amides derived from oleic acid (C18:1 ω9, cis), elaidic acid (C18:1 ω9, trans), eicosenoic acid(s) (C20:1 ω9, cis) and (C20:1 ω9, trans), and the amides and 5′ esters are currently the most preferred derivatives.

Esters, ester-amides and amides of gemcitabine derived from stearic acid (C18:0) and eicosanoic acid (C20:0) are advantageously used in some cases.

Gemcitabine (N4)-elaidic acid amide, gemcitabine-5′-elaidic acid ester and gemcitabine-3′-elaidic acid ester are among the most preferred derivatives, and according to a preferred embodiment of the invention gemcitabine-5′-elaidic acid ester is the active ingredient of the pharmaceutical composition.

In some embodiments, the gemcitabine derivative is gemcitabine-5′-elaidic acid ester (also referred to herein as gemcitabine-5′-elaidate, CP-4055, CP-4126, and CO-101) having the structure of Formula (II):

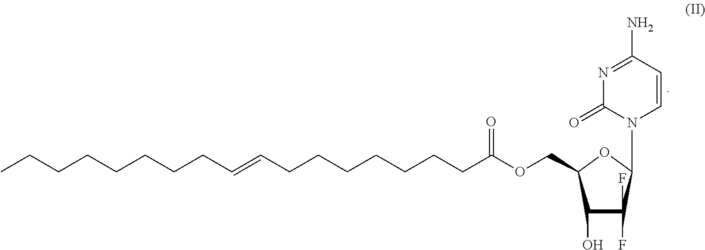

The derivatives of Formula (I) are prepared according to methods known in the prior art. See WO 98/32762 for further details. It is also disclosed in WO 98/32762 that the compounds of Formula (I) are useful in treatment of cancer.

Intake of the lipophilic nucleoside analogs by cells can occur independently of nucleoside transporters.

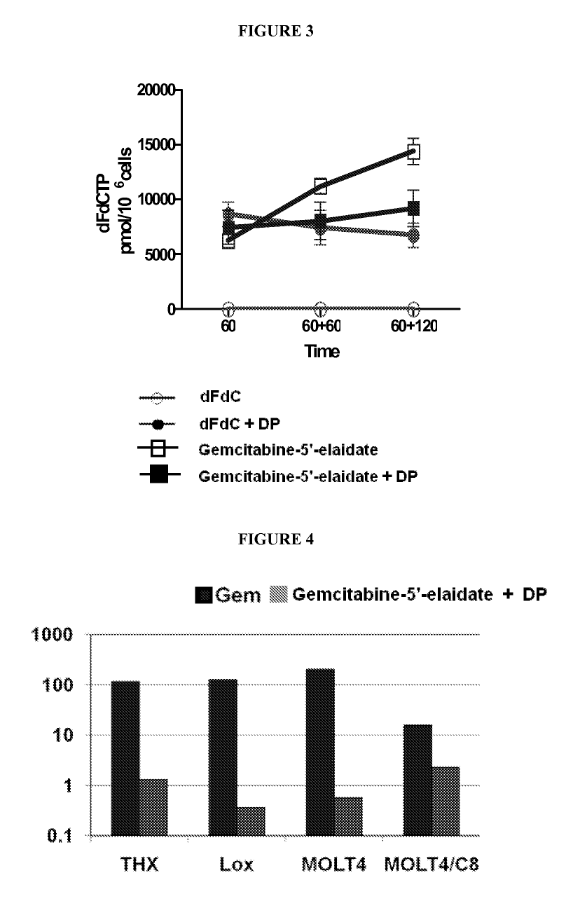

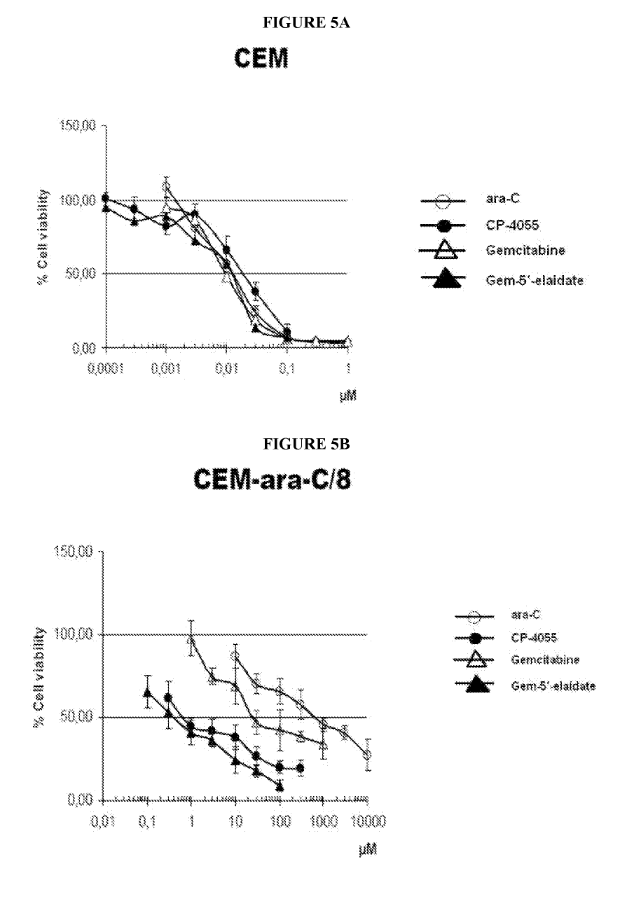

Gemcitabine-5′-elaidic acid ester enters cells in a transporter-independent manner. Gemcitabine triphosphate (dFdCTP) is a highly active metabolite of gemcitabine. It is formed intra-cellularly via the phosphorylation of gemcitabine or gemcitabine-5′-elaidate by deoxycytidine kinase. FIG. 3 illustrates that dFdCTP accumulation in cells is independent of transporters when Gemcitabine-5′-elaidate administered. FIG. 4 illustrates that blocking transporters does not protect against Gemcitabine-5′-elaidate cell killing in vitro. FIGS. 5A and 5B illustrate that Gemcitabine-5′-elaidate kills tumor cells in vitro independent of hENT 1.

Gemcitabine analogs which are not dependant on transporters are effective against some tumor cells that do not respond well to gemcitabine.

C. Treatment with a Single Drug

The methods provided herein are useful in treating cancer or other neoplastic conditions. The term “treatment” as used herein, is intended to encompass administration of gemcitabine analogs prophylactically to prevent or suppress an undesired condition, and therapeutically to eliminate or reduce the extent or symptoms of the condition. Treatment also includes preventing the relapse of an undesired condition, delaying the progression of an undesired condition, and preventing or delaying the onset of an undesired condition. Treatment according to the invention is given to a human or other mammal having a disease or condition creating a need of such treatment. Treatment also includes application of the gemcitabine analogs to cells or organs in vitro. Treatment may be by systemic or local administration.

An effective amount is the amount of active ingredient, e.g., a gemcitabine analog, administered in a single dose or multiple doses necessary to achieve the desired pharmacological effect. A skilled practitioner can determine and optimize an effective dose for an individual patient or to treat an individual condition by routine experimentation and titration well known to the skilled clinician. The actual dose and schedule may vary depending on whether the compositions are administered in combination with other drugs, or depending on inter-individual differences in pharmacokinetics, drug disposition, and metabolism. Similarly, amounts may vary for in vitro applications. It is within the skill in the art to adjust the dose in accordance with the necessities of a particular situation without undue experimentation. Where disclosed herein, dose ranges do not preclude use of a higher or lower dose of a component, as might be warranted in a particular application.

The descriptions of pharmaceutical compositions provided herein include pharmaceutical compositions which are suitable for administration to humans. It will be understood by the skilled artisan, based on this disclosure, that such compositions are generally suitable for administration to any mammal or other animal. Preparation of compositions suitable for administration to various animals is well understood, and the ordinarily skilled veterinary pharmacologist can design and perform such modifications with routine experimentation based on pharmaceutical compositions for administration to humans.

In some embodiments, a single dose of a single chemotherapeutic nucleoside analog is administered to subjects identified by the disclosed methods as having low expression of a nucleoside transporter. In some embodiments, the low expression is in a sample from the subject. In some embodiments, the low expression is in cancer cells. In a preferred embodiment the chemotherapeutic nucleoside analog is gemcitabine-5′-elaidate.

In some embodiments, a chemotherapeutic nucleoside analog may be administered as a single dose. In some embodiments, the single dose composition may be administered once weekly. In some embodiments, the single dose composition may be administered every third day. In some embodiments, the single dose composition may be administered daily for five days. The dosing can be repeated for 1, 2, 3, 4, 5, 6, 7, 8, 9 or 10 weeks.

In some embodiments, a gemcitabine-5′-elaidate may be administered as a single dose. In some embodiments, the single dose composition may be administered once weekly. In some embodiments, the single dose composition may be administered every third day. In some embodiments, the single dose composition may be administered daily for five days. The dosing can be repeated for 1, 2, 3, 4, 5, 6, 7, 8, 9 or 10 weeks.

In some embodiments, a chemotherapeutic nucleoside analog is administered at 25 mg/kg. In some embodiments, a chemotherapeutic nucleoside analog is administered at about 0.01 to 5 mg, 1 to 10 mg, 5 to 20 mg, 10 to 50 mg, 20 to 100 mg, 50 to 150 mg, 100 to 250 mg, 150 to 300 mg, 250 to 500 mg, 300 to 600 mg or 500 to 1000 mg per kg body weight. In some embodiments, a chemotherapeutic nucleoside analog is administered at about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg. In some embodiments, a chemotherapeutic nucleoside analog is administered at about 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 100 mg, 105 mg, 110 mg, 115 mg, 120 mg, 125 mg, 130 mg, 135 mg, 140 mg, 145 mg, 150 mg, 155 mg, or 160 mg.

In some embodiments, gemcitabine-5′-elaidate is administered at 25 mg/kg. In some embodiments, gemcitabine-5′-elaidate is administered at about 0.01 to 5 mg, 1 to 10 mg, 5 to 20 mg, 10 to 50 mg, 20 to 100 mg, 50 to 150 mg, 100 to 250 mg, 150 to 300 mg, 250 to 500 mg, 300 to 600 mg or 500 to 1000 mg per kg body weight. In some embodiments, gemcitabine-5′-elaidate is administered at about 1 mg, 2 mg, 3 mg, 4 mg, 5 mg, 6 mg, 7 mg, 8 mg, 9 mg, 10 mg, 11 mg, 12 mg, 13 mg, 14 mg, 15 mg, 16 mg, 17 mg, 18 mg, 19 mg, 20 mg, 21 mg, 22 mg, 23 mg, 24 mg, 25 mg, 26 mg, 27 mg, 28 mg, 29 mg, 30 mg, 31 mg, 32 mg, 33 mg, 34 mg, 35 mg, 36 mg, 37 mg, 38 mg, 39 mg, 40 mg, 41 mg, 42 mg, 43 mg, 44 mg, 45 mg, 46 mg, 47 mg, 48 mg, 49 mg, or 50 mg. In some embodiments, gemcitabine-5′-elaidate is administered at about 75 mg, 76 mg, 77 mg, 78 mg, 79 mg, 80 mg, 81 mg, 82 mg, 83 mg, 84 mg, 85 mg, 86 mg, 87 mg, 88 mg, 89 mg, 90 mg, 91 mg, 92 mg, 93 mg, 94 mg, 95 mg, 100 mg, 105 mg, 110 mg, 115 mg, 120 mg, 125 mg, 130 mg, 135 mg, 140 mg, 145 mg, 150 mg, 155 mg, or 160 mg.

In some embodiments, the compositions of the invention comprise from 1% to 80% by weight a chemotherapeutic nucleoside analog or a pharmaceutically acceptable salt thereof (such as 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, or 80%.

In some embodiments, the compositions of the invention comprise from 1% to 80% by weight gemcitabine-5′-elaidic acid ester or a pharmaceutically acceptable salt thereof (such as 1%, 1.5%, 2%, 2.5%, 3%, 3.5%, 4%, 4.5%, 5%, 5.5%, 6%, 6.5%, 7%, 7.5%, 8%, 8.5%, 9%, 9.5%, 10%, 10.5%, 11%, 11.5%, 12%, 12.5%, 13%, 13.5%, 14%, 14.5%, 15%, 15.5%, 16%, 16.5%, 17%, 17.5%, 18%, 18.5%, 19%, 19.5%, 20%, 20.5%, 21%, 21.5%, 22%, 22.5%, 23%, 23.5%, 24%, 24.5%, 25%, 25.5%, 26%, 26.5%, 27%, 27.5%, 28%, 28.5%, 29%, 29.5%, 30%, 30.5%, 31%, 31.5%, 32%, 32.5%, 33%, 33.5%, 34%, 34.5%, 35%, 35.5%, 36%, 36.5%, 37%, 37.5%, 38%, 38.5%, 39%, 39.5%, 40%, 40.5%, 41%, 41.5%, 42%, 42.5%, 43%, 43.5%, 44%, 44.5%, 45%, 45.5%, 46%, 46.5%, 47%, 47.5%, 48%, 48.5%, 49%, 49.5%, 50%, 50.5%, 51%, 51.5%, 52%, 52.5%, 53%, 53.5%, 54%, 54.5%, 55%, 55.5%, 56%, 56.5%, 57%, 57.5%, 58%, 58.5%, 59%, 59.5%, 60%, 60.5%, 61%, 61.5%, 62%, 62.5%, 63%, 63.5%, 64%, 64.5%, 65%, 65.5%, 66%, 66.5%, 67%, 67.5%, 68%, 68.5%, 69%, 69.5%, 70%, 70.5%, 71%, 71.5%, 72%, 72.5%, 73%, 73.5%, 74%, 74.5%, 75%, 75.5%, 76%, 76.5%, 77%, 77.5%, 78%, 78.5%, 79%, 79.5%, or 80%.

### D. Combination Therapies

In some embodiments, the level of expression of nucleoside transporter in a sample can direct the administration of two or more therapeutic agents.

In some embodiments, administration of the chemotherapeutic nucleoside analog, gemcitabine analog, or gemcitabine-5′-elaidic acid ester may be combined with the administration of an additional therapeutic agent as part of a therapeutic regimen. The additional therapeutic agent can be administered before, during, or after the administration of the chemotherapeutic nucleoside analog, gemcitabine analog, or gemcitabine-5′-elaidic acid ester. Agents administered during the administration of the chemotherapeutic nucleoside analog, gemcitabine analog, or gemcitabine-5′-elaidic acid ester can be co-administered as a single composition or delivered as part of the same procedure administered at about the same time in separate administration events. Agents administered before or after administration of the gemcitabine analog can be administered in time frames preceding or following gemcitabine analog administration that include the following, without limitation: less than, about, or more than 1, 2, 3, 4, 5, 6, 7, 8, 9, 10, 12, 14, 16, 18, 20, or 22 hours; less than, about, or more than 1, 2, 3, 4, 5, 6, 7, 8, 9, 10, 12, 14 days; less than, about, or more than 1, 2, 3, 4, 5, 6, 7, 8, 9, 10, 12, 14 weeks; less than, about, or more than 1, 2, 3, 4, 5, 6, 7, 8, 9, 10, 12, 14 months; and less than, about, or more than 1, 2, 3, 4, 5, 6, 7, 8, 9, 10, 12, 14 years. In some embodiments, a gemcitabine analog is administered in combination with oxaliplatin. In some embodiments, the additional therapeutic agent is conjugated to the gemcitabine analog. By way of example, it is well known that radioisotopes, drugs, and toxins can be conjugated to antibodies or antibody fragments to facilitate targeting of the radioisotopes, drugs or toxins to tumor sites to enhance their therapeutic efficacy and minimize side effects. Examples of these agents and methods are reviewed in Wawrzynczak and Thorpe (in Introduction to the Cellular and Molecular Biology of Cancer, L. M. Franks and N. M. Teich, eds, Chapter 18, pp. 378-410, Oxford University Press, Oxford, 1986), in Immunoconjugates: Antibody Conjugates in Radioimaging and Therapy of Cancer (C.-W. Vogel, ed., 3-300, Oxford University Press, New York, 1987), in Dillman, R. O. (CRC Critical Reviews in Oncology/Hematology 1:357, CRC Press, Inc., 1984), in Pastan et al. (Cell 47:641, 1986), in Vitetta et al. (Science 238:1098-1104, 1987) and in Brady et al. (Int. J. Rad. Oncol. Biol. Phys. 13:1535-1544, 1987). Other examples of the use of immunoconjugates for cancer and other forms of therapy have been disclosed, inter alia, in Goldenberg, U.S. Pat. Nos. 4,331,647, 4,348,376, 4,361,544, 4,468,457, 4,444,744, 4,460,459, 4,460,561 and 4,624,846, and in Rowland, U.S. Pat. No. 4,046,722, Rodwell et al., U.S. Pat. No. 4,671,958, and Shih et al., U.S. Pat. No. 4,699,784, the disclosures of all of which are incorporated herein in their entireties by reference.

In some embodiments, a transporter blocker is administered before, simultaneously, or after the administration of gemcitabine analogs. The transporter blocker serves to prevent the gemcitabine analogs being pumped by the tumor cells even the gemcitabine analogs may enter the cells. This is because after entering the cells, gemcitabine analog is processed into gemcitabine which could be transported out by one or more the transporters, such hENT 1 and hCNT 1.

In some embodiments, a transporter blocker is administered before, simultaneously, or after the administration of gemcitabine. The transporter blocker will serve to prevent the gemcitabine being pumped by the tumor cells even the gemcitabine may enter the cells. This is because after entering the cells, gemcitabine analog is processed into gemcitabine which could be transported out by one or more the transporters, such hENT1 and hCNT1.

In some embodiments, the transporter blocker is selected from the group consisting of: Acadesine, Acetate, Barbiturates, Benzodiazepines, Calcium Channel Blockers, Carbamazepine, Carisoprodol, Cilostazol, Cyclobenzaprine, Dilazep, Dipyridamole, Estradiol, Ethanol, Flumazenil, Hexobendine, Hydroxyzine, Indomethacin, Inosine, KF24345, Meprobamate, Nitrobenzylthioguanosine, Nitrobenzylthioinosine, Papaverine, Pentoxifylline, Phenothiazines, Phenytoin, Progesterone, Propentofylline, Propofol, Puromycin, R75231, RE 102 BS, Soluflazine, Toyocamycin, Tracazolate, and Tricyclic Antidepressants

In some embodiments, the transporter blocker is administered 20 min, 30 min, 1 hour, 1.5 hour, 2 hour, 3 hours or more after the administration of the gemcitabine or gemcitabine analogs.

In some embodiments, the transporter blocker is administered at a dose of 0.5 to 100 mg/kg. In some embodiments, the transporter blocker is administered at a dose of 1 to 5 mg/kg.

In some embodiments, the gemcitabine analogs is combined with a hedgehog protein inhibitor that is selected from the group consisting of: Cyclopamine, GANT58, and GDC-0449.

In some embodiments, one or more chemotherapeutic agents are combined with anti-tumor or anti-cancer therapeutics capable of decreasing or preventing a further increase in tumor growth. Non-limiting examples are chemotherapeutic agents, cytotoxic agents, and non-peptide small molecules such as Gleevec® (Imatinib Mesylate), Velcade® (bortezomib), Casodex (bicalutamide), Iressa® (gefitinib), and Adriamycin; alkylating agents such as thiotepa and cyclosphosphamide (CYTOXAN™); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, carminomycin, carzinophilin, Casodex™, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elfomithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; PSK®; razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (“Ara-C”); cyclophosphamide; thiotepa; taxanes, e.g., paclitaxel (TAXOL™, Bristol-Myers Squibb Oncology, Princeton, N.J.) and docetaxel (TAXOTERE™, Rhone-Poulenc Rorer, Antony, France); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, (Nolvadex™), raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, and goserelin; chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; camptothecin-11 (CPT-11); topoisomerase inhibitor RFS 2000; difluoromethylornithine (DMFO). Where desired, a compound or pharmaceutical composition described herein can be used in combination with commonly prescribed anti-cancer drugs such as Herceptin®, Avastin®, Erbitux®, Rituxan®, Taxol®, Arimidex®, Taxotere®, ABVD, AVICINE, Abagovomab, Acridine carboxamide, Adecatumumab, 17-N-Allylamino-17-demethoxygeldanamycin, Alpharadin, Alvocidib, 3-Aminopyridine-2-carboxaldehyde thiosemicarbazone, Amonafide, Anthracenedione, Anti-CD22 immunotoxins, Antineoplastic, Antitumorigenic herbs, Apaziquone, Atiprimod, Azathioprine, Belotecan, Bendamustine, BIBW 2992, Biricodar, Brostallicin, Bryostatin, Buthionine sulfoximine, CBV (chemotherapy), Calyculin, cell-cycle nonspecific antineoplastic agents, Dichloroacetic acid, Discodermolide, Elsamitrucin, Enocitabine, Epothilone, Eribulin, Everolimus, Exatecan, Exisulind, Ferruginol, Forodesine, Fosfestrol, ICE chemotherapy regimen, IT-101, Imexon, Imiquimod, Indolocarbazole, Irofulven, Laniquidar, Larotaxel, Lenalidomide, Lucanthone, Lurtotecan, Mafosfamide, Mitozolomide, Nafoxidine, Nedaplatin, Olaparib, Ortataxel, PAC-1, Pawpaw, Pixantrone, Proteasome inhibitor, Rebeccamycin, Resiquimod, Rubitecan, SN-38, Salinosporamide A, Sapacitabine, Stanford V, Swainsonine, Talaporfin, Tariquidar, Tegafur-uracil, Temodar, Tesetaxel, Triplatin tetranitrate, Tris(2-chloroethyl)amine, Troxacitabine, Uramustine, Vadimezan, Vinflunine, ZD6126, and Zosuquidar; Acivicin; Aclarubicin; Acodazole Hydrochloride; Acronine; Adriamycin; Adozelesin; Aldesleukin; Altretamine; Ambomycin; Ametantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Caracemide; Carbetimer; Carboplatin; Carmustine; Carubicin Hydrochloride; Carzelesin; Cedefingol; Chlorambucil; Cirolemycin; Cisplatin; Cladribine; Crisnatol Mesylate; Cyclophosphamide; Cytarabine; Dacarbazine; Dactinomycin; Daunorubicin Hydrochloride; Decitabine; Dexormaplatin; Dezaguanine; Dezaguanine Mesylate; Diaziquone; Docetaxel; Doxorubicin; Doxorubicin Hydrochloride; Droloxifene; Droloxifene Citrate; Dromostanolone Propionate; Duazomycin; Edatrexate; Eflornithine Hydrochloride; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Etoposide; Etoposide Phosphate; Etoprine; Fadrozole Hydrochloride; Fazarabine; Fenretinide; Floxuridine; Fludarabine Phosphate; Fluorouracil; Flurocitabine; Fosquidone; Fostriecin Sodium; Gemcitabine; Gemcitabine Hydrochloride; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-Ib; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansine; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; Peplomycin Sulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Riboprine; Ricin-A; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Sulofenur; Talisomycin; Tecogalan Sodium; Tegafur; Teloxantrone Hydrochloride; Temoporfin; Teniposide; Teroxirone; Testolactone; Tetanus Toxoid; Thiamiprine; Thioguanine; Thiotepa; Tiazofurin; Tirapazamine; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine Sulfate; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; Taxol; thiosemicarbazone derivatives; telomerase inhibitors; arsenic trioxide; planomycin; sulindac sulfide; cyclopamine; purmorphamine; gamma-secretase inhibitors; CXCR4 inhibitors; HH signaling inhibitors; Bmi-1 inhibitors; Bcl-2 inhibitors; Notch-1 inhibitors; DNA checkpoint protein inhibitors; ABC transporter inhibitors; mitotic inhibitors; intercalating antibiotics; growth factor inhibitors; cell cycle modulators; enzymes; topoisomerase inhibitors; biological response modifiers; angiogenesis inhibitors; DNA repair inhibitors; and small G-protein inhibitors. Combinations can be made with one or more than one of the above.

In some aspects gemcitabine or gemcitabine analogs are delivered while a subject is on a low fructose diet. In some embodiments, gemcitabine-5′-elaidic acid ester is delivered while a subject is on a low fructose diet. In some embodiments, gemcitabine-5′-elaidic acid ester is delivered to a subject with low expression of a nucleoside transporter who is on a low fructose diet.

In some aspects gemcitabine or gemcitabine analogs are delivered in conjunction with an inhibitor of fructose metabolism. In some embodiments, gemcitabine-5′-elaidic acid ester is delivered in conjunction with an inhibitor of fructose metabolism. In some embodiments, gemcitabine-5′-elaidic acid ester is delivered to a subject with low expression of a nucleoside transporter in conjunction with an inhibitor of fructose metabolism.

In some embodiments, an individual diagnosed with pancreatic cancer patient is administered a chemotherapeutic nucleoside and zinc. The administered zinc can be at a concentration of 1 μM, 5 μM, 10 μM, 15 μM, 20 μM, 25 μM, 30 μM, 35 μM, to 100 μM. In some embodiments, the individual has been identified as having a high nucleoside transporter expression level. In some embodiments, the nucleoside transporter is hENT1.

In some embodiments, an individual diagnosed with pancreatic cancer patient is administered gemcitabine-5′-elaidic acid ester and zinc. The administered zinc can be at a concentration of 1 μM, 5 μM, 10 μM, 15 μM, 20 μM, 25 μM, 30 μM, 35 μM, to 100 μM. In some embodiments, the individual has been identified as having a low nucleoside transporter expression level. In some embodiments, the nucleoside transporter is hENT 1.

### E. Pharmaceutical Compositions

In some embodiments, a chemotherapeutic nucleoside analog, gemcitabine analog, or gemcitabine-5′-elaidic acid ester described herein is formulated in an aqueous, colloidal suspension. The suspension can be in any concentration known to be useful for injection into a subject, such as 1, 2, 3, 4, 5, 10, 15, 20 or 25 mg/mL.

Various delivery systems are known and can be used to administer a biologically active agent described herein. Non-limiting examples of which include liposomes, microparticles, microcapsules, expression by recombinant cells, receptor-mediated endocytosis (see, e.g., Wu and Wu, (1987), J. Biol. Chem. 262:4429-4432), construction of a therapeutic nucleic acid as part of a retroviral or other vector, liquid suspension, solid or semi-solid compositions, alum precipitations and the like. In some embodiments, a pharmaceutical composition described herein is administered locally to the area in need of treatment. This can be achieved by, for example, and not by way of limitation, local infusion during surgery, by injection, or by means of a catheter. In certain embodiment, the agents are delivered to a tissue comprising cancerous tissue in the subject.

Administration of the selected agent can be effected in one dose, continuously or intermittently throughout the course of treatment. Methods of determining the most effective means and dosage of administration are well known to those of skill in the art and will vary with the composition used for therapy, the purpose of the therapy, the target cell being treated, and the subject being treated. Single or multiple administrations can be carried out with the dose level and pattern being selected by the treating physician or other health care provider. Administration can be tailored to effect one or more of treatment, diagnosis, prognosis, or theranosis of a condition.

A typical daily dosage can range, for example, from about 1 μg/kg to 100 mg/kg or more, 10mg/kg to 100 mg/kg, 20 mg/kg to 80 mg/kg, 20mg/kg to 50 mg/kg, 25 mg/kg to 40 mg/kg, or 60 mg/kg to 80 mg/kg, depending on the factors mentioned above. For repeated administrations over several days or longer, depending on the condition, the treatment is sustained until a desired amelioration of disease occurs. The progress of this therapy is easily monitored by conventional techniques and assays.

As described herein, various dosing regimens are utilized for treatment of cancer. Useful dosing regimens include, but are not limited to, doses of 25 mg/kg administered every third day, 60 mg/kg administered every third day, 80 mg/kg administered every third day subcutaneously; 4 mg/kg administered in a pattern of five consecutive days followed by two days off, 40 mg/kg administered every third day times five, 40 mg/kg administered weekly, 40 mg/kg or 150 mg/kg administered once weekly times two or administered every third day times five; 75 mg/kg/dose administered every third day times four, 5 mg/kg/dose administered daily times five, 1 mg/kg, 4 mg/kg or 75 mg/kg administered as a single dose or administered daily for ten consecutive days, 80 mg/kg administered, intraperitoneally; 7.5, 15, 20, 22.5, 30 or 40 mg/kg administered every three days times five, daily times five or once weekly times two, orally.

In another aspect, methods described herein provide a pharmaceutical composition comprising a) a chemotherapeutic nucleoside analog, gemcitabine analog, or gemcitabine-5′-elaidic acid ester. b) an anti-cancer therapeutic agent, and c) a pharmaceutically acceptable carrier. Non-limiting examples of binding agents and anti-cancer therapeutic agents are described above. The preparation of pharmaceutical compositions is conducted in accordance with generally accepted procedures for the preparation of pharmaceutical preparations. See, for example, Remington: The Science and Practice of Pharmacy, Lippincott Williams & Wilkins; Twenty first Edition (May 1, 2005). Depending on the intended use and mode of administration, an active ingredient is further processed in the preparation of pharmaceutical compositions. Appropriate processing includes, but is not limited to, mixing with appropriate non-toxic and non-interfering components, sterilizing, dividing into dose units, and enclosing in a delivery device.

The active ingredients can be entrapped in microcapsules prepared, for example, by coacervation techniques or by interfacial polymerization, for example, hydroxymethylcellulose or gelatin-microcapsules and poly-(methylmethacylate) microcapsules, respectively, in colloidal drug delivery systems (for example, liposomes, albumin microspheres, microemulsions, nano-particles and nanocapsules) or in macroemulsions. Such techniques are disclosed in Remington: The Science and Practice of Pharmacy, Lippincott Williams & Wilkins; Twenty first Edition (May 1, 2005).

Pharmaceutical compositions for oral, intranasal, or topical administration can be supplied in solid, semi-solid or liquid forms, including tablets, capsules, powders, liquids, and suspensions. Compositions for injection can be supplied as liquid solutions or suspensions, as emulsions, or as solid forms suitable for dissolution or suspension in liquid prior to injection. For administration via the respiratory tract, a preferred composition is one that provides a solid, powder, or aerosol when used with an appropriate aerosolizer device.

Liquid pharmaceutically acceptable compositions can, for example, be prepared by dissolving or dispersing a polypeptide embodied herein in a liquid excipient, such as water, saline, aqueous dextrose, glycerol, or ethanol. The composition can also contain other medicinal agents, pharmaceutical agents, adjuvants, carriers, and auxiliary substances such as wetting or emulsifying agents, and pH buffering agents.

In some embodiments, the compositions comprise one or more excipients, fillers or inert ingredients. In some embodiments, the compositions comprise one or more excipients to aid in the release of the gemcitabine-5′-elaidate or a pharmaceutically acceptable salt thereof In some embodiments, an excipient can be microcrystalline cellulose, sodium carboxymethyl cellulose, sodium starch glycolate, corn starch, colloidal silica, sodium laurel sulphate, magnesium stearate, sodium stearate, silified microcrystalline cellulose (e.g., Prosolve SMCC (HD90)), croscarmellose Sodium, Crospovidone NF, microcrystalline cellulose (e.g., Avicel PH200), or combinations of such excipients. In some embodiments, the wax is a powdered wax that includes linear hydrocarbons such as polyalkalene waxes. In some embodiments, the wax is shellac wax, microcrystalline wax, paraffin-type wax, a polyalkalene glycol, carnauba wax, spermaceti wax, beeswax, candelilla wax, a polyethylene oxide, a hydrogenated vegetable oil, synthetic polyethylene wax, and derivatives or mixtures thereof.

In some embodiments, compositions can be formulated in various dosage forms for oral, parenteral, and topical administration. The compositions can also be formulated as a modified release dosage form, including immediate-, delayed-, extended-, prolonged-, sustained-, pulsatile-, controlled-, extended, accelerated- and fast-, targeted-, programmed-release, and gastric retention dosage forms. These dosage forms can be prepared according to known methods and techniques (see, Remington: The Science and Practice of Pharmacy, supra; Modified-Release Drug Delivery Technology, Rathbone et al., Eds., Drugs and the Pharmaceutical Science, Marcel Dekker, Inc.: New York, N.Y., 2002; Vol. 126, which is herein incorporated by reference in its entirety).

The dosage forms described herein can be manufactured using processes that are well known to those of skill in the art. For example, for the manufacture of tablets, the an effective amount of gemcitabine-5′-elaidate or a pharmaceutically acceptable salt thereof can be dispersed uniformly in one or more excipients, for example, using high shear granulation, low shear granulation, fluid bed granulation, or by blending for direct compression. Excipients include diluents, binders, disintegrants, dispersants, lubricants, glidants, stabilizers, surfactants and colorants. Diluents, also termed “fillers”, can be used to increase the bulk of a tablet so that a practical size is provided for compression. Non-limiting examples of diluents include lactose, cellulose, microcrystalline cellulose, mannitol, dry starch, hydrolyzed starches, powdered sugar, talc, sodium chloride, silicon dioxide, titanium oxide, dicalcium phosphate dihydrate, calcium sulfate, calcium carbonate, alumina and kaolin. Binders can impart cohesion to a composition, such as a tablet formulation and can be used to help a tablet remain intact after compression. Non-limiting examples of suitable binders include starch (including corn starch and pregelatinized starch), gelatin, sugars (e.g., glucose, dextrose, sucrose, lactose and sorbitol), celluloses, polyethylene glycol, waxes, natural and synthetic gums, e.g., acacia, tragacanth, sodium alginate, and synthetic polymers such as polymethacrylates and polyvinylpyrrolidone. Lubricants can also facilitate tablet manufacture; non-limiting examples thereof include magnesium stearate, calcium stearate, stearic acid, glyceryl behenate, and polyethylene glycol. Disintegrants can facilitate tablet disintegration after administration, and non-limiting examples thereof include starches, alginic acid, crosslinked polymers such as, e.g., crosslinked polyvinylpyrrolidone, croscarmellose sodium, potassium or sodium starch glycolate, clays, celluloses, starches, gums and the like. Non-limiting examples of suitable glidants include silicon dioxide, talc and the like. Stabilizers can inhibit or retard drug decomposition reactions, including oxidative reactions. Surfactants can also include and can be anionic, cationic, amphoteric or nonionic. If desired, the tablets can also comprise nontoxic auxiliary substances such as pH buffering agents, preservatives, e.g., antioxidants, wetting or emulsifying agents, solubilizing agents, coating agents, flavoring agents, and the like.

In other embodiments, the compositions of the invention can further comprise suitable additives, including, but not limited to, diluents, binders, surfactants, lubricants, glidants, coating materials, plasticizers, coloring agents, flavoring agents, or pharmaceutically inert materials. Examples of diluents include, for example, cellulose; cellulose derivatives such as microcrystalline cellulose and the like; starch; starch derivatives such as corn starch, cyclodextrin and the like; sugar; sugar alcohol such as lactose, D-mannitol and the like; inorganic diluents such as dried aluminum hydroxide gel, precipitated calcium carbonate, magnesium aluminometasilicate, dibasic calcium phosphate and the like.

Examples of binders include, for example, hydroxypropylcellulose, methylcellulose, hydroxypropylmethylcellulose, povidone, dextrin, pullulane, hydroxypropyl starch, polyvinyl alcohol, scacia, agar, gelatin, tragacanth, macrogol and the like.

Examples of surfactants include, for example, sucrose esters of fatty acids, polyoxyl stearate, polyoxyethylene hydrogenated castor oil, polyoxyethylene polyoxypropylene glycol, sorbitan sesquioleate, sorbitan trioleate, sorbitan monostearate, sorbitan monopalmitate, sorbitan monolaurate, polysorbate, glyceryl monostearate, sodium lauryl sulfate, lauromacrogol and the like.

Examples of lubricants include, for example, stearic acid, calcium stearate, magnesium stearate, talc and the like.

Examples of glidants include, for example, dried aluminum hydroxide gel, magnesium silicate and the like.

Examples of coating materials include, for example, hydroxypropylmethyl cellulose 2910, aminoalkyl methacrylate copolymer E, polyvinylacetal diethylaminoacetate, macrogol 6000, titanium oxide and the like. Examples of plasticizers include, for example, triethyl citrate, triacetin, macrogol 6000 and the like.

A pharmaceutically acceptable salt includes, but is not limited to, a metal salt, such as a sodium salt, a potassium salt, and a lithium salt; an alkaline earth metal salt, such as a calcium salt, a magnesium salt, and the like; an organic amine salt, such as a triethylamine salt, a pyridine salt, a picoline salt, an ethanolamine salt, a triethanolamine salt, a dicyclohexylamine salt, a N,N′-dibenzylethylenediamine salt, and the like; an inorganic acid salt such as a hydrochloride salt, a hydrobromide salt, a sulfate salt, a phosphate salt, and the like; an organic acid salt such as a formate salt, an acetate salt, a trifluoroacetate salt, a maleate salt, a tartrate salt, and the like; a sulfonate salt such as a methanesulfonate salt, a benzenesulfonate salt, p-toluenesulfonate salt, and the like; and an amino acid salt, such as an arginate salt, an asparginate salt, a glutamate salt, and the like.

Additional pharmaceutically acceptable salts of include bitartrate, bitartrate hydrate, hydrochloride, p-toluenesulfonate, phosphate, sulfate, trifluoroacetate, bitartrate hemipentahydrate, pentafluoropropionate, hydrobromide, mucate, oleate, phosphate dibasic, phosphate monobasic, acetate trihydrate, bis(heptafuorobutyrate), bis(pentaflu oropropionate), bis(pyridine carboxylate), bis(trifluoroacetate), chlorhydrate, sulfate pentahydrate, thiosemicarbazone, p-nitrophenylhydrazone, o-methyloxime, semicarbazone, bis (methylcarbamate), amsonate(4,4-diaminostilbene-2,2-disulfonate), benzenesulfonate, benzonate, bicarbonate, bisulfate, bitartrate, borate, butyrate, calcium edetate, camphorsulfonate, camsylate, carbonate, citrate, clavulariate, dihydrochloride, edetate, edisylate, estolate, esylate, fiunarate, fumarate, gluceptate, gluconate, glutamate, glycollylarsanilate, hexafluorophosphate, hexylresorcinate, hydrabamine, hydrobromide, hydrochloride, hydroxynaphthoate, iodide, isothionate, lactate, lactobionate, laurate, malate, maleate, mandelate, mesylate, methylbromide, methylnitrate, methylsulfate, mucate, napsylate, nitrate, N-methylglucamine ammonium salt, 3-hydroxy-2-naphthoate, oleate, oxalate, palmitate, pamoate (1,1-methene-bis-2-hydroxy-3-naphthoate, einbonate), pantothenate, phosphate/diphosphate, picrate, polygalacturonate, propionate, p-toluenesulfonate, salicylate, stearate, subacetate, succinate, sulfate, sulfosaliculate, suramate, tannate, tartrate, teoclate, tosylate, triethiodide, valerate salts and hydrates.

For parenteral administration, the gemcitabine analogs can be formulated in a unit dosage injectable form (solution, suspension, emulsion) in association with a pharmaceutically acceptable parenteral vehicle. Such vehicles are inherently nontoxic, and non-therapeutic. Examples of such vehicles are water, saline, Ringer's solution, dextrose solution, and 5% human serum albumin. Nonaqueous vehicles such as fixed oils and ethyl oleate can also be used. Liposomes can be used as carriers. The vehicle can contain minor amounts of additives such as substances that enhance isotonicity and chemical stability, e.g., buffers and preservatives. The antibodies will typically be formulated in such vehicles at concentrations of about 1 mg/ml to 10 mg/ml.

Where desired, the pharmaceutical compositions can be formulated in slow release or sustained release forms, whereby a relatively consistent level of the active compound are provided over an extended period. Suitable examples of sustained-release preparations include semipermeable matrices of solid hydrophobic polymers containing the antibody, which matrices are in the form of shaped articles, e.g., films, or microcapsules. Examples of sustained-release matrices include polyesters, hydrogels (for example, poly(2-hydroxyethyl-methacrylate), or poly(vinylalcohol)), polylactides (U.S. Pat. No. 3,773,919), copolymers of L-glutamic acid and γ ethyl-L-glutamate, non-degradable ethylene-vinyl acetate, degradable lactic acid-glycolic acid copolymers such as the LUPRON DEPOT® (injectable microspheres composed of lactic acid-glycolic acid copolymer and leuprolide acetate), and poly-D-(−)-3-hydroxybutyric acid.

### F. Treatment Regime

The treatment duration and regimen can vary depending on the particular condition and subject that is to be treated. For instance, chemotherapeutic nucleoside analog, gemcitabine analog, or gemcitabine-5′-elaidic acid ester can be administered by the subject method over at least 1, 7, 14, 30, 60, 90 days, or a period of months, years, or even throughout the lifetime of a subject. Treatment can also be designed to reach a suitable positive outcome, non-limiting examples of which include partial remission, complete remission, a reduction in tumor size, stable tumor size, slowing of tumor growth, reducing the frequency of metastasis, prevention of metastasis for a period exceeding at least about 3 months, at least about 6 months, at least about 9 months or at least about 12 months, extension of expected life expectancy, prevention of recurrence of a cancer, extension of the expected time necessary for recurrence of cancer, and reducing the frequency or severity of one or more sequelae of cancer, such as pain, edema, prevention of the disease, etc.

Repeated administrations of chemotherapeutic nucleoside analog, gemcitabine analog, or gemcitabine-5′-elaidic acid ester can also be administered at time intervals indicated based on the non-limiting factors listed above, as can be determined by one skilled in the art. For example, gemcitabine analog can be administered every day, 2×, 3×, 4× or 5× daily, every other day, every third day, every fourth day, every fifth day, 2, 3, 4, 5 or 6 days a week, every week, every two weeks, every three weeks, every four weeks, or every five weeks. The fixed doses, or predicted dose needed, can, for example, continue to be administered until disease progression, adverse event, or other time as determined by a physician or other health care provider is reached. For example, from about two, three, or four, up to about 20 or more fixed doses can be administered. In some embodiments, one or more loading dose(s) of the binding agent are administered, followed by one or more maintenance dose(s) of the binding agent. In some embodiments, a plurality of the same fixed dose is administered to the subject.

Methods of delivery of compositions described herein include but are not limited to intra-arterial, intra-muscular, intravenous, intranasal, subcutaneous, intraperitoneal, intracerobrospinal, intra-articular, intrasynovial, intrathecal, intratumoral, intradermal, intracerebral, peritumoral, anal, vaginal, and oral routes. In some embodiments, a composition described herein is administered intraperitoneally. In some embodiments, a composition described herein is administered intravenously. In some embodiments, a composition described herein is administered orally.

The terms “effective amount”, pharmacologically effective amount“, “physiologically effective amount” or “therapeutically effective amount,” as used herein, refer to a sufficient amount of a therapeutic agent or a compound being administered which will relieve to some extent one or more of the symptoms of the disease or condition being treated. The result is reduction and/or alleviation of the signs, symptoms, or causes of a disease, or any other desired alteration of a biological system. For example, an “effective amount” for therapeutic uses is the amount of the composition that includes a therapeutic agent herein required to provide a desired level of therapeutic agent in the bloodstream or at the site of action (e.g., the sinuses, or the lung tissue) of a subject to be treated and thereby produce a clinically significant decrease in disease symptoms. The precise amount will depend upon numerous factors, e.g., the specific therapeutic agent, the activity of the therapeutic agent, the delivery device employed, the physical characteristics of the therapeutic agent, intended use by the subject (i.e., the number of doses administered per day), subject considerations, and the like, and can readily be determined by one skilled in the art, based upon the information provided herein. In some instances, an appropriate “effective” amount in any individual case is determined using techniques, such as a dose escalation study.

### G. Targeted Cancer Therapy

Identification of biologically effective doses (BED), the dose or dose range that maximally inhibits the intended target, beyond which dose escalation is likely to add toxicity without benefit, allows for improved treatment. Moreover, many agents are used in combination with cytotoxic therapies, where added toxicity may not be tolerable, further supporting BED-based dosing. “Targeted-therapy” implies that populations of likely responders exists, and can be identified.

Successful diagnostic targeting of a nucleoside transporter protein complex determines if tumor growth or survival can be ameliorated using a particular gemcitabine analog, what a preferred dosing regimen can be, as well as possible combination therapies.

Methods described herein are capable of better assessing the expected efficacy of a proposed therapeutic agent (or combination of agents) for each individual. Methods described herein are advantageous for the additional reasons that they are both time and cost effective in assessing the efficacy of chemotherapeutic regimens and are minimally traumatic to cancer patients.

In some embodiments, administration of a gemcitabine analog provides greater therapeutic benefit, such as remission of tumor, to a hENT1-low expressing patient.

### IX. Diagnostic Kits

In one aspect, provided herein are kits that can be used in the above-described methods. In some embodiments, a kit comprises a composition described herein in one or more containers. In some embodiments, provided herein are kits comprising gemcitabine analogs. The binding agents can be, without limitation, any of those described above. The binding agents can further be provided, without limitation, in any of the formulations and/or doses described above. The kits can further comprise additional agents, such as those described above, or as an anti-cancer agent used in combination with a gemcitabine analog. The agents can be provided in any suitable container, including but not limited to test tubes, vials, flasks, bottles, ampules, syringes, or the like. The agents can be provided in a form that can be directly administered to a subject, or in a form that requires preparation prior to administration, such as in the reconstitution of lyophilized agents. Agents can be provided in aliquots of single-doses or as stocks from which multiple doses can be obtained.

Provided herein are kits for characterizing a mammalian tumor's responsiveness to a Gemcitabine analog therapy, where the kits include an antibody that binds hENT1. Further, the kit can include additional components other then the above-identified antibodies, including but not limited to additional antibodies. Such kits can be used, for example, by a clinician or physician as an aid to selecting an appropriate therapy for a particular patient, for example, a cancer patient under consideration for Gemcitabine analog-directed therapy.

In some embodiment patients are treated based upon information, such as genomic information, that is obtained from or about the patient's ancestors. In some embodiments, the patients are treated based upon racial population data. In some embodiments, the kits of the present invention include means for collecting genomic information from family members or questionnaires which query for ancestral information.

All publications and patent documents cited herein are incorporated herein by reference as if each such publication or document was specifically and individually indicated to be incorporated herein by reference. Citation of publications and patent documents is not intended as an admission that any is pertinent prior art, nor does it constitute any admission as to the contents or date of the same. The invention having now been described by way of written description, those of skill in the art will recognize that the invention can be practiced in a variety of embodiments and that the foregoing description and examples below are for purposes of illustration and not limitation of the claims that follow.

### EXAMPLES

While alternative embodiments have been shown and described herein, it will be obvious to those skilled in the art that such embodiments are provided by way of example only and are not to be construed as limiting upon the present invention. Numerous variations, changes, and substitutions will now occur to those skilled in the art without departing from the invention. It should be understood that various alternatives to the embodiments of the invention described herein can be employed in practicing the invention. It is intended that the following claims define the scope of the invention and that methods and structures within the scope of these claims and their equivalents be covered thereby.

### Example 1

### Schedule Dependency of Antitumor Activity in TAX-II-1 Xenograft

The antitumor activity of gemcitabine is schedule-dependent. Nonclinical treatment with a 3 day interval is superior to schedules with daily or weekly injections. Therefore, a study determines whether such a schedule-dependency exists for a pharmaceutical formulation of the gemcitabine analog, gemcitabine-5′-elaidic acid ester.

The human tumor xenograft TAX-II-1, a malignant fibrous sarcoma, is grown subcutaneously in female Balb/C mice. The compounds are administered intraperitoneally. Prior to the main study, dose-finding studies are performed to determine the maximum tolerated dose of gemcitabine and gemcitabine-5′-elaidic acid ester (3 mice/dose group) in the daily and weekly schedules.

### Example 2

Antitumor Activity of Gemcitabine-5′-elaidic Acid Ester in Two Human Pancreas Tumor Models

A human pancreatic carcinoma cell line MiaPaCa-2 is implanted subcutaneously in nude Balb/C mice and another human pancreatic carcinoma cell line Panc-1 is implanted in female NCr nude mice, respectively. Treatment starts when the tumors reach a mean diameter of 6 mm. The treatment schedule is intraperitoneal on Days 0, 3, 6, 9, and 12 with gemcitabine doses of 120 and 60 mg/kg or gemcitabine-5′-elaidic acid ester doses of 80 and 40 mg/kg.

### Example 3

Antitumor Activity of Gemcitabine-5′-elaidic Acid Ester in Human Glioma U373 Grown Subcutaneously

Male, NMRI mice are implanted subcutaneously with human glioma cells U373 (Day 0) and are treated with gemcitabine-5′-elaidic acid ester or gemcitabine, intraperitoneally, once daily on Days 10-14, 17-21, 24-28, and 31-35. Cyclophosphamide (intraperitoneally, Day 10) is used as positive control.

### Example 4

Antitumor Activity of Gemcitabine-5′-elaidic Acid Ester in Human Glioma U373 Grown Intracerebrally

NMRI male mice are inoculated with human glioma cells U373 intracerebrally. The tumors at the injection site (at the surface of the brain), as well as the intracerebral tumors, are evaluated. Gemcitabine-5′-elaidic acid ester is administered intraperitoneally on Days 4-8, 11-15, 18-22, and 25-29 and is active at 4 mg/kg per injection. Gemcitabine-5′-elaidic acid ester is active intracerebrally after intraperitoneal administration.

### Example 5

Antitumor Activity of Gemcitabine-5′-elaidic Acid Ester and Gemcitabine in Human Glioma Xenograft Model U373

NCR mice are inoculated subcutaneously on Day 0 with human glioma cells U373. Treatment (intraperitoneal) starts on Day 7, either every third day or weekly. The effect of gemcitabine-5′-elaidic acid ester is better than that of gemcitabine when dosed Days 7 and 14. In the every third day schedule, the effect of gemcitabine-5′-elaidic acid ester is equal to gemcitabine.

### Example 6

Antitumor Activity of Gemcitabine-5′-elaidic Acid Ester in Human Colon Xenograft Models Co5776 and Co6044

Fragments of human colon cancers Co5776 and Co6044 are implanted subcutaneously in NCR female mice (7-8 mice/group). Gemcitabine (120 mg/kg) and gemcitabine-5′-elaidic acid ester (40 mg/kg) are administered intraperitoneally, every third day for five times. The treatment starts on Day 8 when the tumors reach a mean volume of 100 mm³ Significant antitumor effect is observed in both colon cancers following treatment with gemcitabine or gemcitabine-5′-elaidic acid ester and the activity of both compounds is similar.

### Example 7

Schedule-dependency of Gemcitabine-5′-elaidic Acid Ester in Human Colon Cancer Xenograft Co5776

NCr female mice implanted subcutaneously with human colon carcinoma fragments, Co5776, are treated intraperitoneally. Two different schedules of gemcitabine-5′-elaidic acid ester are administered, once weekly times 2 (Days 11 and 18), and every third day times 5 (Days 11, 14, 17, 20, and 23). Antitumor activity of gemcitabine-5′-elaidic acid ester is similar for 150 mg/kg once weekly, administered twice or 40 mg/kg every third day administered five times.

### Example 8

Antitumor Effect of Gemcitabine-5′-elaidic Acid Ester in P388 Murine Leukemia Model

B6D2F1 female mice are injected intraperitoneally with P388 leukemia cells and treated intraperitoneally with gemcitabine-5′-elaidic acid ester or gemcitabine at two different schedules. The compounds are administered either as single doses or daily for 10 consecutive days. White blood cell (WBC) and thrombocyte counts are determined on Days 4 and 11. The WBC count and thrombocyte counts are relatively low in the control animals on Day 4 due to the tumor model. Gemcitabine-5′-elaidic acid ester induces significant antitumor activity at all the tested doses and schedules.

### Example 9

Antitumor Effect of Gemcitabine-5′-elaidic Acid Ester in Co-26 Liver Metastasis Model

Antitumor effect of single dose of gemcitabine-5′-elaidic acid ester or gemcitabine is tested in Balb/C female mice injected with Co-26 murine colon cancer cells in the spleen (a liver metastasis model). Gemcitabine-5′-elaidic acid ester shows significant antitumor activity at a dose of 75 mg/kg with a significant increase in mean survival both in days and in Treated/Control (T/C, %) over control animals treated with saline. A moderate reduction in the hematological parameters is seen. The activity of gemcitabine is similar.

### Example 10

Antitumor Activity of Gemcitabine-5′-elaidic Acid Ester in Murine Lewis Lung

Murine Lewis Lung cells are intravenously injected to BDF1 mice. Mean survival as well as body weight change and tumor free survivors on Day 72 are endpoints of the study. Cyclophosphamide (CTX), and 1-β-D-arabinofuranosylcytosine (Ara-C) are used as controls. Treatment is administered once daily intraperitoneally on Days 1-4 and 7-11. CTX is intravenously administered on Day 1 and highly active and non-toxic.

### Example 11

### Antitumor Activity in Human Colon Cancer Co6044

Oral administration of Gemcitabine-5′-elaidic acid ester and gemcitabine is tested in NCr:nu/nu mice for potential antitumor activity. Treatment is every third day repeated five times. In a follow-up experiment, the antitumor activity of oral and intraperitoneal treatment is compared in this colon cancer model (Co6044). The doses for oral treatment with Gemcitabine-5′-elaidic acid ester and gemcitabine are lower (20, 30, or 40 mg/kg) than in the previous experiment.

### Example 12

Oral Activity and Schedule Dependency of Gemcitabine-5′-elaidic Acid Ester in Mice with Co6044

In this experiment, the antitumor activity of oral Gemcitabine-5′-elaidic acid ester in three different administration schedules is compared: every 3 days times 5, daily times 5, and once weekly times 2. Three doses are tested per schedule. High antitumor activity is obtained with all the tested schedules. The activity observed is dose dependent.

### Example 13

Antitumor Activity in Human Non-Small Cell Lung Cancers MAKSAK and EKVX Following Oral Administration of Gemcitabine-5′-elaidic Acid Ester

Oral activity of Gemcitabine-5′-elaidic acid ester is determined in Balb/C female mice implanted with tumor fragments of human NSCLC lines MAKSAK and EKVX. Tumor fragments of 2-3 mm are implanted subcutaneously on each flank of the mice. Treatment starts when the average tumor diameter reaches 6 mm (Day 0). Gemcitabine or Gemcitabine-5′-elaidic acid ester is diluted in 0.9% saline to obtain a final administration volume of 0.1 mL/10 g. Gemcitabine-5′-elaidic acid ester or gemcitabine is administered either Days 0, 3, 6, 9, and 12 or daily on Days 0-4.

### Example 14

### Independency of Nucleoside Transporter and Intracellular Triphosphate Levels

hENT1 can inhibited in vitro by various agents including dipyridamole, NBMPR, and NBI. In order to test whether gemcitabine-5′-elaidate entry into the cells is independent of a nucleoside transporter such as hENT1, dipyridamole is used to block hENT1. The intracellular level of dFdCTP is determined in CEM cells after 60 minutes exposure to either dFdC or gemcitabine-5′-elaidate, with or without the addition of dipyridamole (DP). The compounds are administered at 10 μM, and the triphosphates are determined using HPLC. Deamination is blocked using tetrahydrouridine. The triphosphate level after exposure to gemcitabine with DP is below level of detection. In the absence of DP, the level of triphosphate decreases with time after exposure to gemcitabine. The maximum dFdCTP concentration is reached at the last time point measured after gemcitabine-5′-elaidate exposure, and the levels obtained with inhibitor (DP) present are even increased compared to exposure without inhibitor.

### Example 15

Gemcitabine-5′-elaidate Combination Studies In Vitro

The cell lines A549 human NSCLC and WiDr human colon carcinoma are used to determine the combination index and also the effect of Gemcitabine-5′-elaidate on the cell cycle. A sulforhodamine B colorimetric assay is used to determine growth inhibition after 72 hours. Pemetrexed combinations are tested using the MTT assay. Combinations with pemetrexed are also tested in the WiDr-LF cell line, which are WiDr cells cultured under low folate conditions. The IC₂₅ and IC₅₀ values (inhibitory concentrations at 25% or 50%, respectively) are calculated. The IC₂₅ value is used for combinations with serial dilutions of oxaliplatin or docetaxel, and the IC₅₀ value is used for pemetrexed combinations. Median effect analysis is performed.

The combination of Gemcitabine-5′-elaidate with oxaliplatin is synergistic in both tumor cell lines; whereas, the combination with docetaxel is antagonistic. Antagonistic activity is observed for the combination Gemcitabine-5′-elaidate and pemetrexed, although under low folate conditions the antagonistic activity is slightly reduced.

The effect on DNA platinum accumulation in DNA after exposure to oxaliplatin and oxaliplatin combined with Gemcitabine-5′-elaidate (oxaliplatin [200 μM] combined with Gemcitabine-5′-elaidate [0.004 μM]) is also determined. An increase of platinum adducts is observed in the WiDr cell line but not in A549.

The effect on cell cycle is determined after 72 h exposure using flow cytometry combined with propidium iodide staining For cell cycle analysis, the concentration range is from 0.0005 μM to 0.05 μM in the A549 cell line and from 0.001 μM to 0.1 μM in the WiDr cell line. In the A549 cell line, no effect on cell cycle is observed at the tested concentrations. In the WiDr cell line, Gemcitabine-5′-elaidate causes an S phase accumulation at the two highest concentrations and a dose dependent sub G1 accumulation. The effect on cell cycle is obtained also for the combinations with pemetrexed. In the A549 cell line, the G0/G1 increased from 8.8% to 14.1%, and the S phase from 23.1% to 28.5%. In the WiDr cell line only a marginal increase in the G2/M phase is observed.

### Example 16

In Vivo Study of Gemcitabine-5′-elaidic Acid Ester in Patients with Advanced Pancreatic Cancer.

Uptake of hydrophilic nucleoside analogs into tumor cells is determined by membrane transporter expression. Clinical response to gemcitabine is correlated with expression of the nucleoside transporter hENT1. Gemcitabine-5′-elaidic acid ester is, by virtue of a fatty acid conjugate, can enter cells in a transporter-independent manner. This study compares gemcitabine-5′-elaidic acid ester with gemcitabine and correlates activity with tumor cell hENT1 expression in pancreatic cancer patients.

Treatment-naive patients with advanced or metastatic pancreatic adenocarcinoma, ECOG performance status<2, and adequate haematologic, renal and hepatic function are eligible. The study has a two-stage design—a pilot stage in which all patients receive gemcitabine-5′-elaidic acid ester, followed by a randomized stage comparing gemcitabine-5′-elaidic acid ester with gemcitabine (1:1). Both agents are administered as a 30-min IV infusion on days (d) 1, 8, 15 every 4 weeks. The doses of gemcitabine-5′-elaidic acid ester and gemcitabine are 1250 mg/m2/d and 1000 mg/m2/d, respectively. hENT1 expression on tumor cells (biopsies from primary or metastasis) is determined by immunohistochemistry. Patients receive treatment in the range from 1 to 6 cycles. All patients are evaluated using Response Evaluation Criteria In Solid Tumors (RECIST) criteria.

### Example 17

Gemcitabine-5′-elaidate Enters Tumor Cells Independent of hENT1

Drugs: Gemcitabine-5′-elaidate was provided by Clavis Pharma (Oslo, Norway), tetra hydro uridine (THU) was from Calbiochem (Merck, Darmstadt, Germany), dipyridamole and ara-C were from Sigma-Aldrich (St. Louis, Mo., USA) and dFdC was from Eli-Lilly (Indianapolis, Ind., USA). The radioactively labeled drugs were obtained from Moravek (Brea, Calif., USA); the drugs were labeled with tritium on the 5-C site of the base. A mix of radioactive and non-radioactive compounds was made for exposure of cells; dFdC and CP-4126 were used at a final concentration of 8.9 μM and a specific activity of 586 and 391 mCi/mmol, respectively.

Cell lines: For the experiments the CCRF-CEM human leukemia cell line and its dCK negative variant (CEM/dCK−) were used. The cell lines were cultured in RPMI medium (BioWhittaker, Verviers, Belgium) supplemented with 10% fetal calf serum (PAA laboratories, Pasching, Austria) and HEPES buffer (BioWhittaker). Of the nucleoside influx transporters, the CEM cell line only expresses the equilibrative nucleoside transporter (hENT) and not the concentrative nucleoside transporter (hCNT). Belt et al. Adv. Enzyme Regul., 1993, 33, 235-52. Dipyridamole (DP) was used to inhibit the influx of the drugs by hENT. In order to get a clean picture, THU was also added to inhibit deamination of ara-C and dFdC.

In situ tracing: The method was based on a procedure described earlier. Peters et al., Eur. J. Cancer Clin. Oncol., 1984, 20, 1425-31. Shortly, cells were harvested and resuspended in fresh medium at 5×10⁶ cells/ml. Of this cell suspension 100 μl was used for each experiment. To inhibit deamination by CDA, THU was added at a final concentration of 100 μM. Yusa et al., Biochem. Biophys. Res. Commun, 1995, 206, 486-9; the drugs were added to reach a final concentration of 8.9 μM for dFdC/CP-4126, respectively. The cells were incubated for 0, 30 and 60 minutes at 37° C. To measure drug retention the drug containing medium was replaced after 60 minutes and the cells were incubated for 60 minutes in drug free medium. Thereafter the cells were spun down (3000 g, 2 min, 4° C.) and the medium was stored as extracellular fraction at −20° C. The cells were washed with cold PBS (12000 g, 1 min, 4° C.). The cell pellet was resuspended in 45 cold PBS and extracted by addition of 5 μl perchloric acid (5 M) and chilled on ice for 20 minutes. After spinning down (12000 g, 3 min, 4° C.) the perchloric acid pellet containing the precipitated nucleic acids was resuspended in 200 μl NaOH (1 M). The supernatant containing the cytosolic fraction was neutralized with 10 μl KH₂PO₄ (5 M) and stored as intracellular fraction at −20° C.

Of the extracellular and intracellular cytosolic samples 5 μl was spotted on a plastic backed silica TLC plate (Merck KgaA, Darmstadt, Germany). The chromatography was performed with 3:2 chloroform/methanol as a mobile phase. After separation the spots were visualized with UV light and cut into separate scintillation vials, and radioactivity was eluted by overnight incubation in methanol. The samples were measured together with the perchloric acid pellet samples in an LSC counter.

Intracellular localization: Intracellular localization of the lipophilic analogs was investigated using a ProteoExtract™ Subcellular Proteome Extraction Kit (Calbiochem). Cells were incubated with 8.9 μM of the radioactive drugs as described above and deamination was inhibited by 100 μM THU. The samples were incubated for 60 minutes at 37° C. After incubation the cells were washed and with the different reagents provided with the kit; the samples were separated into subcellular fractions: a cytosolic, a membrane, a nuclear and a cytoskeletal fraction.

Triphosphate accumulation: Cells were treated with 1 and 10 μM of dFdC, while for the lipophilic analogs 10 and 100 μM was used. The cells were incubated for 60 minutes and retention of the triphosphates was investigated after 60 and 120 minutes incubation in drug-free medium. Dipyridamole was added to inhibit drug influx by the hENT transporter. After incubation the cell pellet was resuspended in ice-cold PBS and incubated for 20 minutes at 4° C. with 40% trichloroacetic acid. After centrifugation (10,000 g, 10 min, 4° C.) the supernatant was treated with a 2-fold excess of trioctylamine/1,1,2-trichlorotrifluorethane (1:4) and spun down (10,000 g, 1 min) and the aqueous phase was stored at −20° C. until analysis by HPLC on a Whatman Partisphere SAX column (GE healthcare, Chalfont St. Giles, UK) using gradient (dFdC-TP) elution as described earlier. Noordhuis et al., Leuk. Res., 1996, 20, 127-34; Ruiz van Haperen, et al., Biochem. Pharmacol., 1994, 48, 1327-39.

Results: Since the perchloric acid procedure not only precipitated dFdC incorporated into DNA, but also additional CP-4126, this precluded measurement of dFdC incorporation into DNA after exposure to CP-4126. In order to get more insight in the formation of active metabolites formed from the prodrugs, it was therefore determined the accumulation of triphosphates (and dFdC-TP). After 60 minutes incubation with dFdC, more triphosphates were formed than after incubation with CP-4126, respectively (FIG. 5A). The concentration of dFdC-TP from the parent compounds decreased after washing away the drugs but the concentration of dFdC-TP from CP-4126 was retained at a similar level. Dipyridamole completely inhibited entry of the parent compounds, abolishing accumulation of dFdC-TP. However, dipyridamole even increased the concentration of triphosphates from the prodrugs even after washing away the drugs.

### Example 18

### Survival of Pancreatic Patients Receiving Gemcitabine Monotherapy

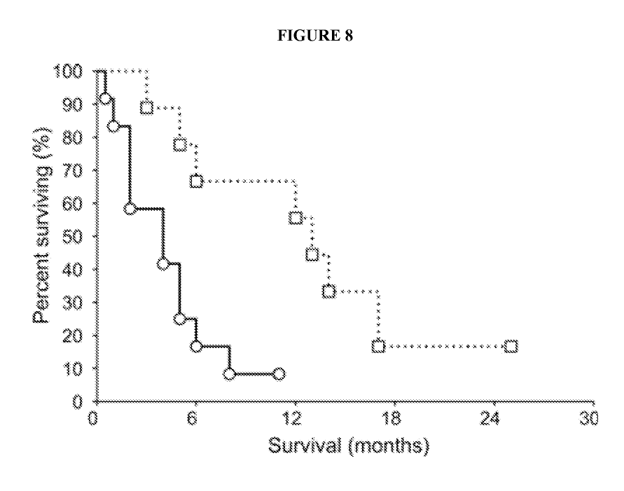

Patients suffering from pancreatic cancer are administered gemcitabine monotherapy. Post mortem studies of the patients' tumors determine the levels of hENT1 expression in the tumors. FIG. 8 depicts a Kaplan-Meier plot of survival for patients receiving the gemcitabine monotherapy Patients with high hENT1 expression (dotted line) outperform those with areas of hENT1-low tumor.

### Example 19

Treatment of a Pancreatic Cancer Patient with a Gemcitabine Analog Based on a Determination of hENT1 Levels in Cancerous Cells

An individual is diagnosed with pancreatic cancer. The patient provides a sample. The sample contains cancerous cells from the patient. The sample comes from a biopsy of the tumor, from circulating tumor cells, or from another source.

The sample is tested to determine the level of expression of hENT1 or another nucleoside transporter. The level is detected by immunohistochemistry, by determining the amount of mRNA relevant to hENT1 in the sample, or by other known means.

The testing of the sample takes place at a testing facility. The physician or his staff ships the sample to this facility. The sample is fixed or frozen for shipment. The sample is mixed with an antibody prior to shipment and analyzed when it arrives at the facility or the sample is shipped with no antibody added prior to shipment.

The nucleoside transporter level is compared to a control. The control is non-cancerous cells from the patient or is samples from another population. The control samples are analyzed at the same time as the sample or the analysis is based upon computer stored data where the comparison is done by using a rule based upon previously collected and characterized control data. This comparison allows for the classification of the sample as a “high,” “low,” or “normal,” sample based upon nucleoside transporter levels.

The information regarding the patient's nucleoside transporter levels is transmitted to the physician. This transmission optionally includes a recommendation regarding which chemotherapeutic nucleoside analog the patient should be treated with. Patients with low expression of hENT1 receive a recommendation to use a hydrophobic chemotherapeutic nucleoside analog such as gemcitabine-5′-elaidic acid ester.

A patient with hENT1 low expression receives a script from the physician for gemcitabine-5′-elaidic acid ester. The gemcitabine-5′-elaidic acid ester includes dosage instructions for the patient which are based upon a low level of hENT1. The gemcitabine-5′-elaidic acid ester includes indications based upon low hENT1 expression.

The patient takes the gemcitabine-5′-elaidic acid ester and responds favorably to the treatment.

While preferred embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention. It is intended that the following claims define the scope of the invention and that methods and structures within the scope of these claims and their equivalents be covered thereby.

1. A method of ameliorating a cancer comprising the steps of:
a) detecting hENT1 expression level in the subject and comparing the hENT1 expression level in the subject with a control level of hENT1 expression level; and
b) administering an effective dose of gemcitabine-5′-elaidate in the range of 20 mg/kg to 80 mg/kg to ameliorate the cancer in the subject exhibiting a decreased level of hENT1 expression.

2. The method of claim 1, wherein the control level of hENT1 expression has previously been determined from a source other than the subject.

3. The method of claim 1, wherein the control level of hENT1 is contemporaneously determined from a source other than the subject.

4. The method of claim 1, wherein the control level is determined by obtaining a second non-cancerous sample from the subject.

5. The method of claim 1, wherein the control level is determined by obtaining a non-cancerous sample from a different subject.

6. The method of claim 1, wherein the control level is determined using the level of hENT1 expression in multiple control sources.

7. The method of claim 6, wherein the control level is determined by obtaining a statistical distribution of hENT1 levels.

8. The method of claim 1, wherein the control level is determined from cultured cells engineered to express hENT1.

9. The method of claim 1, wherein the control level is determined from cells engineered to not express hENT1.

10. The method of claim 1, wherein the control level is a clinically accepted reference level.

11. The method of claim 1, wherein the level of hENT1 expression in the subject is classified as high, medium or low according to an H-Score.

12. The method of claim 11, wherein the level of hENT1 expression in the subject is classified as a low sample when the H-Score is less than or equal to the overall median H-Score.

13. The method of claim 1, wherein the effective dose of gemcitabine-5′-elaidate is administered as a single dose or as multiple doses.

14. The method of claim 1, wherein the effective dose is administered every day, every third day, every third day times four, every third day times five, daily for ten consecutive days, or once weekly.

15. The method of claim 1, wherein the effective dose is administered in a regimen selected from (i) doses of 25 mg/kg administered every third day, (ii) doses of 60 mg/kg administered every third day, (iii) doses of 80 mg/kg administered every third day, (iv) 40 mg/kg administered every third day times five, (v) 40 mg/kg administered weekly, (vi) 40 mg/kg administered once weekly times two or administered every third day times five, (vii) 75 mg/kg/dose administered every third day times four, (viii) 75 mg/kg administered as a single dose or administered daily for ten consecutive days, (ix) 80 mg/kg administered intraperitoneally, (x) 20, 22.5, 30 or 40 mg/kg administered every three days times five, daily times five or once weekly times two, orally, and combinations thereof.

16. The method of claim 1, wherein the effective dose is administered intravenously, subcutaneously, orally or a combination thereof.

17. The method of claim 1, wherein the subject is human.

18. The method of claim 1, wherein the subject is non-responsive, less responsive or has stopped responding to treatment with a chemotherapeutic agent.

19. The method of claim 18, wherein the chemotherapeutic agent is gemcitabine.

20. The method of claim 1, wherein the cancer is renal cancer, including renal cell carcinoma, glioblastoma, brain tumors, chronic or acute leukemias including acute lymphocytic leukemia (ALL), adult T-cell leukemia (T-ALL), chronic myeloid leukemia, acute lymphoblastic leukemia, chronic lymphocytic leukemia, lymphomas including Hodgkin's and non-Hodgkin's lymphoma, lymphocytic lymphoma, primary CNS lymphoma, T-cell lymphoma, Burkitt's lymphoma, anaplastic large-cell lymphomas (ALCL), cutaneous T-cell lymphomas, nodular small cleaved-cell lymphomas, peripheral T-cell lymphomas, Lennert's lymphomas, immunoblastic lymphomas, T-cell leukemia/lymphomas (ATLL), entroblastic/centrocytic (cb/cc) follicular lymphomas cancers, diffuse large cell lymphomas of B lineage, angioimmunoblastic lymphadenopathy (AILD)-like T cell lymphoma and HIV associated body cavity based lymphomas), embryonal carcinomas, undifferentiated carcinomas of the rhino-pharynx (e.g., Schmincke's tumor), Castleman's disease, Kaposi's Sarcoma, multiple myeloma, Waldenstrom's macroglobulinemia and other B-cell lymphomas, nasopharangeal carcinomas, bone cancer, skin cancer, cancer of the head or neck, cutaneous or intraocular malignant melanoma, uterine cancer, rectal cancer, cancer of the anal region, stomach cancer, testicular cancer, uterine cancer, carcinoma of the fallopian tubes, carcinoma of the endometrium, carcinoma of the cervix, carcinoma of the vagina, carcinoma of the vulva, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, solid tumors of childhood, cancer of the bladder, cancer of the kidney or ureter, carcinoma of the renal pelvis, neoplasm of the central nervous system (CNS), tumor angiogenesis, spinal axis tumor, brain stem glioma, pituitary adenoma, epidermoid cancer, squamous cell cancer, environmentally induced cancers including those induced by asbestos, e.g., mesothelioma or a combinations of said cancers.

21. The method of claim 1, wherein the cancer is non-small cell lung cancer (NSCLC), sarcoma, malignant melanoma, prostate cancer, breast cancer, pancreatic cancer, colon cancer including a colon carcinoma, glioma, leukemia, or liver cancer.

22. The method of claim 1, wherein the gemcitabine-5′-elaidate is administered in combination with one or more additional chemotherapeutic or cytotoxic agents.

23. The method of claim 1, wherein the gemcitabine-5′-elaidate is administered at an effective dose in the range of 20 mg/kg to 50 mg/kg.
